(19) United States
(12) Patent Application Publication
(10) Pub. No.: US 2020/0330967 A1
(43) Pub. Date: Oct. 22, 2020

(54) MAGNETIC MACROPOROUS POLYMERIC HYBRID SCAFFOLDS FOR IMMOBILIZING BIONANOCATALYSTS

(71) Applicant: ZYMtronix Catalytic Systems, Inc., Ithaca, NY (US)

(72) Inventors: Stephane Cedric Corgie, Ithaca, NY (US); Ricki Chairil, Monterey Park, CA (US)

(73) Assignee: ZYMtronix Catalytic Systems, Inc., Ithaca, NY (US)

(21) Appl. No.: 16/092,211

(22) PCT Filed: Apr. 5, 2017
(86) PCT No.: PCT/US17/26086

§ 371 Date: Oct. 8, 2018

(51) Int. Cl. B01J 31/00; B01J 31/06; B01J 35/00; B01J 35/02; B01J 35/10; B01J 37/36; C12N 11/04; C12N 11/084; C12N 11/14

(52) U.S. Cl. CPC B01J 31/003; B01J 31/069; B01J 35/0013; B01J 35/0033; C12N 11/14; B01J 35/1076; B01J 37/36; C12N 11/04; C12N 11/084; B01J 35/023

(57) ABSTRACT

The present invention provides magnetic macroporous polymeric hybrid scaffolds for supporting and enhancing the effectiveness of bionanocatalysts (BNC). The novel scaffolds comprise cross-linked water-insoluble polymers and an approximately uniform distribution of embedded magnetic microparticles (MMP). The cross-linked polymer comprises polyvinyl alcohol (PVA) and optionally additional polymeric materials. The scaffolds may take any shape by using a cast during preparation of the scaffolds. Alternatively, the scaffolds may be ground to microparticles for use in biocatalytic reactions. Alternatively, the scaffolds may be shaped as beads for use in biocatalyst reactions. Methods for preparing and using the scaffolds are also provided.

### CROSS REFERENCE TO RELATED APPLICATIONS

This application claims the benefit of U.S. Provisional Application No. 62/323,663, filed Apr. 16, 2016, which is incorporated herein by reference in its entirety.

### FIELD OF THE INVENTION

The present invention provides magnetic macroporous polymeric hybrid scaffolds for supporting and enhancing the effectiveness of bionanocatalysts (BNC). The novel scaffolds comprise cross-linked water-insoluble polymers and an approximately uniform distribution of embedded magnetic microparticles (MMP). The cross-linked polymer comprises polyvinyl alcohol (PVA) and optionally additional polymeric materials. The scaffolds may take any shape by using a cast during preparation of the scaffolds. In certain embodiments, the scaffolds may be shaped as beads for use in biocatalyst reactions. In alternative embodiments, the scaffolds may be ground to microparticles for use in biocatalytic reactions. Methods for preparing and using the scaffolds are also provided.

### BACKGROUND OF THE INVENTION

Magnetic enzyme immobilization involves the entrapment of enzymes in mesoporous magnetic clusters that self-assemble around the enzymes. The immobilization efficiency depends on a number of factors that include the initial concentrations of enzymes and nanoparticles, the nature of the enzyme surface, the electrostatic potential of the enzyme, the nature of the nanoparticle surface, and the time of contact. Enzymes used for industrial purposes in biocatalytic processes should be highly efficient, stable before and during the process, reusable over several biocatalytic cycles, and economical.

Mesoporous aggregates of magnetic nanoparticles may be incorporated into continuous or particulate macroporous scaffolds. The scaffolds may or may not be magnetic. Such scaffolds are discussed in WO2014/055853 and Corgie et al., Chem. Today 34(5):15-20 (2016), incorporated by reference herein in its entirety.

### SUMMARY OF THE INVENTION

The present invention provides magnetic macroporous polymeric hybrid scaffolds for supporting and enhancing the effectiveness of bionanocatalysts (BNC). The novel scaffolds comprise cross-linked water-insoluble polymers and an approximately uniform distribution of embedded magnetic microparticles (MMP). The cross-linked polymer comprises polyvinyl alcohol (PVA) and optionally additional polymeric materials. The scaffolds may take any shape by using a cast during preparation of the scaffolds. Alternatively, the scaffolds may be ground to microparticles for use in biocatalyst reactions. Alternatively, the scaffolds may be shaped as beads for use in biocatalyst reactions. Methods for preparing and using the scaffolds are also provided.

Thus, the invention provides a magnetic macroporous polymeric hybrid scaffold comprising a cross-linked water-insoluble polymer and an approximately uniform distribution of embedded magnetic microparticles (MMP). The polymer comprises at least polyvinyl alcohol (PVA), has MMPs of about 50-500 nm in size, pores of about 1 to about 50 μm in size, about 20% to 95% w/w MMP, wherein the scaffold comprises an effective surface area for incorporating bionanocatalysts (BNC) that is about total 1-15 m²/g; wherein the total effective surface area for incorporating the enzymes is about 50 to 200 m²/g; wherein said scaffold has a bulk density of between about 0.01 and about 10 g/ml; and wherein said scaffold has a mass magnetic susceptibility of about 1.0×10⁻³ to about 1×10⁻⁴ m³ kg⁻¹. In a preferred embodiment, the magnetic macroporous polymeric hybrid scaffold comprises a contact angle for the scaffold with water that is about 0-90 degrees.

In preferred embodiments, the cross-linked water-insoluble polymer is essentially polyvinyl alcohol (PVA). In more preferred embodiments, the scaffold further comprises a polymer selected from the group consisting of polyethylene, polypropylene, poly-styrene, polyacrylic acid, polyacrylate salt, polymethacrylic acid, polymethacrylate salt, polymethyl methacrylate, polyvinyl acetate, polyvinylfluoride, polyvinylidenefluoride, polytetrafluoroethylene, a phenolic resin, a resorcinol formaldehyde resin, a polyamide, a polyurethane, a polyester, a polyimide, a polybenzimidazole, cellulose, hemicellulose, carboxymethyl cellulose (CMC), 2-hydroxyethylcellulose (HEC), ethylhydroxyethyl cellulose (EHEC), xylan, chitosan, inulin, dextran, agarose, alginic acid, sodium alginate, polylactic acid, polyglycolic acid. a polysiloxane, a polydimethylsiloxane, and a polyphosphazene.

In other more preferred embodiments, the magnetic macroporous polymeric hybrid scaffold comprises PVA and CMC, PVA and alginate, PVA and HEC, or PVA and EHEC.

In some embodiments, the magnetic macroporous polymeric hybrid scaffold is formed in the shape of a monolith. In other embodiments, the scaffold is formed in a shape suited for a particular biocatalytic process. In other embodiments, the scaffold is in the form of a powder, wherein said powder comprises particles of about 150 to about 1000 μm in size.

The invention provides the magnetic macroporous polymeric hybrid scaffold as disclosed herein, further comprising a bionanocatalyst (BNC). In some embodiments, the BNC comprises a magnetic nanoparticle (MNP) and an enzyme selected from the group consisting of hydrolases, hydroxylases, hydrogen peroxide producing enzymes (HPP), nitralases, hydratases, dehydrogenases, transaminases, ene reductases (EREDS), imine reductases (IREDS), oxidases, oxidoreductases, peroxidases, oxynitrilases, isomerases, and lipases.

The invention provides a method of preparing a water-insoluble macroporous polymeric hybrid scaffold, comprising mixing a water-soluble polymer with water and magnetic microparticles (MMP) to form a suspension of about 3 to 50 cP; adding a cross-linking reagent to said mixture; ultra-sonicating said mixture; freezing said mixture at a temperature of about −200 to 0 degrees Celsius; freeze drying said mixture; and cross-linking said water-soluble polymer; wherein said cross-linking step results in water-insoluble polymers.

In some embodiments, the method the cross-linking step is accomplished by exposure to ultraviolet light, heating the mixture at a temperature of about 60 to 500 degrees Celsius, or a combination thereof. In preferred embodiments, the method further comprises the step of applying a magnetic field after the ultra-sonication step to organize the MMPs by alignment of the magnetic moments of said MMPs.

In some embodiments of the method, the water-soluble polymer is polyvinyl alcohol (PVA). In other embodiments, the water-soluble polymer further comprises a polymer selected from the group consisting of polyethylene, polypropylene, poly-styrene, polyacrylic acid, polyacrylate salt, polymethacrylic acid, polymethacrylate salt, polymethyl methacrylate, polyvinyl acetate, polyvinylfluoride, polyvinylidenefluoride, polytetrafluoroethylene, a phenolic resin, a resorcinol formaldehyde resin, a polyamide, a polyurethane, a polyester, a polyimide, a polybenzimidazole, cellulose, hemicellulose, carboxymethyl cellulose (CMC), 2-hydroxyethylcellulose, ethylhydroxyethyl cellulose, xylan, chitosan, inulin, dextran, agarose, alginic acid, sodium alginate, polylactic acid, polyglycolic acid. a polysiloxane, a polydimethylsiloxane, and a polyphosphazene.

In more preferred embodiments, the polymers comprise PVA and CMC, PVA and alginate, PVA and HEC, or PVA and EHEC.

In some embodiments, the cross-linking reagent is selected from the group consisting of citric acid, all calcium salts, 1,2,3,4-butanetetracarboxylic acid (BTCA), glutaraldehyde, and poly(ethylene glycol). In a preferred embodiment, the cross-linking reagent is citric acid.

In some embodiments, the freezing step results in a water-soluble macroporous polymeric hybrid scaffold that is in the shape of a monolith. In other embodiments, the freezing step results in a water-soluble macroporous polymeric hybrid scaffold that is in a shape suited for a particular biocatalytic process. In other embodiments, the water-insoluble macroporous polymeric hybrid scaffold is ground into a powder of about 10 to about 1000 μm in size.

The invention provides a method of catalyzing a reaction between a plurality of substrates, comprising exposing the substrates to the magnetic macroporous polymeric hybrid scaffold under conditions in which the BNC catalyzes the reaction between the substrates. In preferred embodiments, the reaction is used in the manufacture of a pharmaceutical product, medicament, food product, garment, detergent, a fuel product, a biochemical product, a paper product, or a plastic product.

Some embodiments of the invention provides a method for forming water-insoluble macroporous polymeric hybrid scaffolds that are shaped into beads of about 500 to about 5000 μm in size.

In another embodiment, the invention provides a method of catalyzing a reaction between a plurality of substrates, comprising exposing the substrates to the the magnetic macroporous polymeric hybrid scaffold under conditions in which the BNC catalyzes the reaction between the substrates and the reaction is used in a process for removing a contaminant from a solution. In a preferred embodiment, the solution is an aqueous solution.

### BRIEF DESCRIPTION OF THE DRAWINGS

FIG. 1 shows an exemplary block diagram of the magnetic scaffold production process.

FIG. 2A shows a scanning electron micrograph (SEM) image of magnetic scaffold MO32 (1.875 g magnetite, 3.125 mL 10% poly(vinyl alcohol), 3.125 mL 2% low-viscosity carboxymethylcellulose (CMC), and 13.75 mL excess water).

FIG. 2B shows an SEM image of magnetic scaffold MO32-50-hi μ (1.875 g magnetite, 3.125 mL 10% poly(vinyl alcohol), 3.125 mL 2% high-viscosity carboxymethylcellulose (CMC), and 43.75 mL excess water).

FIG. 3A shows an SEM image of magnetic scaffold MO32 (1.875 g magnetite, 3.125 mL 10% poly(vinyl alcohol), 3.125 mL 2% low-viscosity carboxymethylcellulose (CMC), and 13.75 mL excess water), containing 83% magnetite by dry solid mass.

FIG. 3B shows SEM image of failed magnetic scaffold MO48 (0.90 g magnetite, 11 mL 10% poly(vinyl alcohol), 3.71 mL 6% low-viscosity carboxymethylcellulose (CMC), and 23.2 mL excess water), which contained 40% magnetite by dry solid mass.

FIG. 4A shows an SEM image of magnetic scaffold MO32-40 (1.875 g magnetite, 3.125 mL 10% poly(vinyl alcohol), 3.125 mL 2% low-viscosity carboxymethylcellulose (CMC), and 33.75 mL excess water), containing 83% magnetite by dry solid mass, frozen while applying a uniform magnetic field of about 2G, perpendicular to the liquid nitrogen bath.

FIG. 4B shows an SEM image of magnetic scaffold MO32-40 (1.875 g magnetite, 3.125 mL 10% poly(vinyl alcohol), 3.125 mL 2% low-viscosity carboxymethylcellulose (CMC), and 33.75 mL excess water), containing 83% magnetite by dry solid mass, frozen while applying a uniform magnetic field of about 2G, parallel to the liquid nitrogen bath.

FIG. 5 demonstrates the reduced surface fouling potential of the scaffolds as opposed to ordinary magnetite powder.

FIG. 6A shows the activity of immobilized nitrilase as measured fluorometrically with the ammonia quantification method. Three samples are compared: (1) free nitrilase; (2) BMC composed of pH 6 nitrilase/pH 3 magnetite nanoparticle BNCs with 20% loading templated on magnetic macroporous polymeric hybrid scaffold MO32-40; and (3) BMC composed of pH 6 nitrilase/pH 3 magnetite nanoparticle BNCs with 20% loading templated on simple magnetite powder (50-100 nm) with 9.5% final effective loading.

FIG. 6B shows the activity of immobilized w-transaminase as measured spectrophotometrically with acetophenone absorbance at 245 nm. Three samples are compared: (1) free w-transaminase; (2) BMC composed of pH 7.15 ω-transaminase/pH 3 magnetite nanoparticle BNCs with 20% loading templated on magnetic macroporous polymeric hybrid scaffold MO32-40; and (3) BMC composed of pH 7.15 w-transaminase/pH 3 magnetite nanoparticle BNCs with 20% loading templated on simple magnetite powder (50-100 nm) with 6.2% effective loading. Because enzyme immobilization efficiency was below 100% for the simple magnetite powder, uncaptured enzyme was removed and replaced with the appropriate amount of water to eliminate the contribution of free enzyme to the immobilized enzyme results.

FIG. 6C shows the activity of immobilized carbonic anhydrase measured by fluorometric pH-based method. Three samples are compared: (1) free carbonic anhydrase; (2) BMC composed of pH 6 carbonic anhydrase/pH 11 magnetite nanoparticle BNCs with 20% loading templated on magnetic macroporous polymeric hybrid scaffold MO32-40; and (3) BMC composed of pH 6 carbonic anhydrase/pH 11 magnetite nanoparticle BNCs with 20% loading templated on simple magnetite powder (50-100 nm) with 9.5% effective loading.

FIG. 6D shows the activity of immobilized horseradish peroxidase as measured spectrophotometrically with quinoneimine dye complex absorbance at 500 nm. Three samples are showed: (1) free horseradish peroxidase (HRP); (2) BMC composed of pH 5 horseradish peroxidase/pH 11 magnetite nanoparticle BNCs with 5% loading templated on magnetic macroporous polymeric hybrid scaffold MO32-40; and BMC composed of pH 5 horseradish peroxidase/pH 11 magnetite nanoparticle BNCs with 5% loading templated on simple magnetite powder (50-100 nm) with 3% effective loading.

FIG. 7 shows immobilized and non-immobilized chloroperoxidase (CPO) activity. The biocatalytic conversion of (R)-limonene to (1S,2S,4R)-(+)-limonene-1,2-diol was measured spectrophotometrically at 490 nm using adrenochrome reporter reaction.

FIG. 8 Shows immobilized and free lipase activity. Biocatalytic conversion of p-nitrophenol laurate to p-nitrophenol and laurate was measured spectrophotometrically at 314 nm at pH 4.

### DETAILED DESCRIPTION OF THE INVENTION

The present invention provides compositions and methods for supporting and enhancing the effectiveness of BNC's. This is accomplished, for the first time, using the magnetic macroporous polymeric hybrid scaffolds disclosed herein. The novel scaffolds comprise cross-linked water-insoluble polymers and an approximately uniform distribution of embedded magnetic microparticles (MMP). The cross-linked polymer comprises polyvinyl alcohol (PVA) and optionally additional polymeric materials. The scaffolds may take any shape by using a cast during preparation of the scaffolds. Alternatively, the scaffolds may be ground to macroparticles and sieved to defined sizes for biocatalytic reactions. Methods for preparing and using the scaffolds are also provided.

Self-assembled mesoporous nanoclusters comprising entrapped enzymes are highly active and robust. The technology is a powerful blend of biochemistry, nanotechnology, and bioengineering at three integrated levels of organization: Level 1 is the self-assembly of enzymes with magnetic nanoparticles (MNP) for the synthesis of magnetic mesoporous nanoclusters. This level uses a mechanism of molecular self-entrapment to immobilize and stabilize enzymes. Level 2 is the stabilization of the MNPs into other matrices. Level 3 is product conditioning and packaging for Level 1+2 delivery. The assembly of magnetic nanoparticles adsorbed to enzyme is herein also referred to as a “bionanocatalyst” (BNC).

MNPs allow for a broader range of operating conditions such as temperature, ionic strength and pH. (The size and magnetization of the MNPs affect the formation and structure of the NPs, all of which have a significant impact on the activity of the entrapped enzymes. By virtue of their surprising resilience under various reaction conditions, MNPs can be used as improved enzymatic or catalytic agents where other such agents are currently used. Furthermore, they can be used in other applications where enzymes have not yet been considered or found applicable.

The BNC contains mesopores that are interstitial spaces between the magnetic nanoparticles. The enzymes are preferably embedded or immobilized within at least a portion of mesopores of the BNC. As used herein, the term “magnetic” encompasses all types of useful magnetic characteristics, including permanent magnetic, superparamagnetic, paramagnetic, ferromagnetic, and ferrimagnetic behaviors.

The magnetic nanoparticle or BNC has a size in the nanoscale, i.e., generally no more than 500 nm. As used herein, the term “size” can refer to a diameter of the magnetic nanoparticle when the magnetic nanoparticle is approximately or substantially spherical. In a case where the magnetic nanoparticle is not approximately or substantially spherical (e.g., substantially ovoid or irregular), the term “size” can refer to either the longest the dimension or an average of the three dimensions of the magnetic nanoparticle. The term “size” may also refer to an average of sizes over a population of magnetic nanoparticles (i.e., “average size”).

In different embodiments, the magnetic nanoparticle has a size of precisely, about, up to, or less than, for example, 500 nm, 400 nm, 300 nm, 200 nm, 100 nm, 50 nm, 40 nm, 30 nm, 25 nm, 20 nm, 15 nm, 10 nm, 5 nm, 4 nm, 3 nm, 2 nm, or 1 nm, or a size within a range bounded by any two of the foregoing exemplary sizes.

In the BNC, the individual magnetic nanoparticles can be considered to be primary nanoparticles (i.e., primary crystallites) having any of the sizes provided above. The aggregates of nanoparticles in a BNC are larger in size than the nanoparticles and generally have a size (i.e., secondary size) of at least about 5 nm. In different embodiments, the aggregates have a size of precisely, about, at least, above, up to, or less than, for example, 5 nm, 8 nm, 10 nm, 12 nm, 15 nm, 20 nm, 25 nm, 30 nm, 35 nm, 40 nm, 45 nm, 50 nm, 60 nm, 70 nm, 80 nm, 90 nm, 100 nm, 150 nm, 200 nm, 300 nm, 400 nm, 500 nm, 600 nm, 700 nm, or 800 nm, or a size within a range bounded by any two of the foregoing exemplary sizes.

Typically, the primary and/or aggregated magnetic nanoparticles or BNCs thereof have a distribution of sizes, i.e., they are generally dispersed in size, either narrowly or broadly dispersed. In different embodiments, any range of primary or aggregate sizes can constitute a major or minor proportion of the total range of primary or aggregate sizes. For example, in some embodiments, a particular range of primary particle sizes (for example, at least about 1, 2, 3, 5, or 10 nm and up to about 15, 20, 25, 30, 35, 40, 45, or 50 nm) or a particular range of aggregate particle sizes (for example, at least about 5, 10, 15, or 20 nm and up to about 50, 100, 150, 200, 250, or 300 nm) constitutes at least or above about 50%, 60%, 70%, 80%, 90%, 95%, 98%, 99%, or 100% of the total range of primary particle sizes. In other embodiments, a particular range of primary particle sizes (for example, less than about 1, 2, 3, 5, or 10 nm, or above about 15, 20, 25, 30, 35, 40, 45, or 50 nm) or a particular range of aggregate particle sizes (for example, less than about 20, 10, or 5 nm, or above about 25, 50, 100, 150, 200, 250, or 300 nm) constitutes no more than or less than about 50%, 40%, 30%, 20%, 10%, 5%, 2%, 1%, 0.5%, or 0.1% of the total range of primary particle sizes.

The aggregates of magnetic nanoparticles (i.e., “aggregates”) or BNCs thereof can have any degree of porosity, including a substantial lack of porosity depending upon the quantity of individual primary crystallites they are made of. In particular embodiments, the aggregates are mesoporous by containing interstitial mesopores (i.e., mesopores located between primary magnetic nanoparticles, formed by packing arrangements). The mesopores are generally at least 2 nm and up to 50 nm in size. In different embodiments, the mesopores can have a pore size of precisely or about, for example, 2, 3, 4, 5, 10, 12, 15, 20, 25, 30, 35, 40, 45, or 50 nm, or a pore size within a range bounded by any two of the foregoing exemplary pore sizes. Similar to the case of particle sizes, the mesopores typically have a distribution of sizes, i.e., they are generally dispersed in size, either narrowly or broadly dispersed. In different embodiments, any range of mesopore sizes can constitute a major or minor proportion of the total range of mesopore sizes or of the total pore volume. For example, in some embodiments, a particular range of mesopore sizes (for example, at least about 2, 3, or 5, and up to 8, 10, 15, 20, 25, or 30 nm) constitutes at least or above about 50%, 60%, 70%, 80%, 90%, 95%, 98%, 99%, or 100% of the total range of mesopore sizes or of the total pore volume. In other embodiments, a particular range of mesopore sizes (for example, less than about 2, 3, 4, or 5 nm, or above about 10, 15, 20, 25, 30, 35, 40, 45, or 50 nm) constitutes no more than or less than about 50%, 40%, 30%, 20%, 10%, 5%, 2%, 1%, 0.5%, or 0.1% of the total range of mesopore sizes or of the total pore volume.

The magnetic nanoparticles can have any of the compositions known in the art. In some embodiments, the magnetic nanoparticles are or include a zerovalent metallic portion that is magnetic. Some examples of such zerovalent metals include cobalt, nickel, and iron, and their mixtures and alloys. In other embodiments, the magnetic nanoparticles are or include an oxide of a magnetic metal, such as an oxide of cobalt, nickel, or iron, or a mixture thereof. In some embodiments, the magnetic nanoparticles possess distinct core and surface portions. For example, the magnetic nanoparticles may have a core portion composed of elemental iron, cobalt, or nickel and a surface portion composed of a passivating layer, such as a metal oxide or a noble metal coating, such as a layer of gold, platinum, palladium, or silver. In other embodiments, metal oxide magnetic nanoparticles or aggregates thereof are coated with a layer of a noble metal coating. The noble metal coating may, for example, reduce the number of charges on the magnetic nanoparticle surface, which may beneficially increase dispersibility in solution and better control the size of the BNCs. The noble metal coating protects the magnetic nanoparticles against oxidation, solubilization by leaching or by chelation when chelating organic acids, such as citrate, malonate, or tartrate are used in the biochemical reactions or processes. The passivating layer can have any suitable thickness, and particularly, at least, up to, or less than, about for example, 0.1 nm, 0.2 nm, 0.3 nm, 0.4 nm, 0.5 nm, 0.6 nm, 0.7 nm, 0.8 nm, 0.9 nm, 1 nm, 2 nm, 3 nm, 4 nm, 5 nm, 6 nm, 7 nm, 8 nm, 9 nm, or 10 nm, or a thickness in a range bounded by any two of these values.

Magnetic materials useful for the invention are well-known in the art. Non-limiting examples comprise ferromagnetic and ferromagnetic materials including ores such as iron ore (magnetite or lodestone), cobalt, and nickel. In other embodiments, rare earth magnets are used. Non-limiting examples include neodymium, gadolinium, sysprosium, samarium-cobalt, neodymium-iron-boron, and the like. In yet further embodiments, the magnets comprise composite materials. Non-limiting examples include ceramic, ferrite, and alnico magnets. In preferred embodiments, the magnetic nanoparticles have an iron oxide composition. The iron oxide composition can be any of the magnetic or superparamagnetic iron oxide compositions known in the art, e.g., magnetite (Fe₃O₄), hematite (α-Fe₂O₃), maghemite (γ-Fe₂O₃), or a spinel ferrite according to the formula AB₂O₄, wherein A is a divalent metal (e.g., Xn²⁺, Ni²⁺, Mn²⁺, Co²⁺, Ba²⁺, Sr²⁺, or combination thereof) and B is a trivalent metal (e.g., Fe³⁺, Cr³⁺, or combination thereof).

The individual magnetic nanoparticles or aggregates thereof or BNCs thereof possess any suitable degree of magnetism. For example, the magnetic nanoparticles, BNCs, or BNC scaffold assemblies can possess a saturated magnetization (Ms) of at least or up to about 5, 10, 15, 20, 25, 30, 40, 45, 50, 60, 70, 80, 90, or 100 emu/g. The magnetic nanoparticles, BNCs, or BNC-scaffold assemblies preferably possess a permanent magnetization (Mr) of no more than (i.e., up to) or less than 5 emu/g, and more preferably, up to or less than 4 emu/g, 3 emu/g, 2 emu/g, 1 emu/g, 0.5 emu/g, or 0.1 emu/g. The surface magnetic field of the magnetic nanoparticles, BNCs, or BNC-scaffold assemblies can be about or at least, for example, about 0.5, 1, 5, 10, 50, 100, 200, 300, 400, 500, 600, 700, 800, 900, or 1000 Gauss (G), or a magnetic field within a range bounded by any two of the foregoing values. If microparticles are included, the microparticles may also possess any of the above magnetic strengths.

The magnetic nanoparticles or aggregates thereof can be made to adsorb a suitable amount of enzyme, up to or below a saturation level, depending on the application, to produce the resulting BNC. In different embodiments, the magnetic nanoparticles or aggregates thereof may adsorb about, at least, up to, or less than, for example, 1, 5, 10, 15, 20, 25, or 30 pmol/m2 of enzyme. Alternatively, the magnetic nanoparticles or aggregates thereof may adsorb an amount of enzyme that is about, at least, up to, or less than, for example, about 10%, 20%, 30%, 40%, 50%, 60%, 70%, 80%, 90%, or 100% of a saturation level.

The magnetic nanoparticles or aggregates thereof or BNCs thereof possess any suitable pore volume. For example, the magnetic nanoparticles or aggregates thereof can possess a pore volume of about, at least, up to, or less than, for example, about 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1 cm3/g, or a pore volume within a range bounded by any two of the foregoing values.

The magnetic nanoparticles or aggregates thereof or BNCs thereof possess any suitable specific surface area. For example, the magnetic nanoparticles or aggregates thereof can have a specific surface area of about, at least, up to, or less than, for example, about 50, 60, 70, 80, 90, 100, 110, 120, 130, 140, 150, 160, 170, 180, 190, or 200 m 2/g.

MNPs, their structures, organizations, suitable enzymes, and uses are described in WO2012122437 and WO2014055853, incorporated by reference herein in their entirety.

Some embodiments of the invention comprise hydrolases. Hydrolases catalyze the hydrolysis of many types of chemical bonds by using water as a substrate. The substrates typically have hydrogen and hydroxyl groups at the site of the broken bonds. Hydrolases are classified as EC 3 in the EC number classification of enzymes. Hydrolases can be further classified into several subclasses, based upon the bonds they act upon. Exemplary hydrolases and the bonds they hydrolyze include EC 3.1: ester bonds (esterases: nucleases, phosphodiesterases, lipase, phosphatase), EC 3.2: sugars (DNA glycosylases, glycoside hydrolase), EC 3.3: ether bonds, EC 3.4: peptide bonds (Proteases/peptidases), EC 3.5: carbon-nitrogen bonds, other than peptide bonds, EC 3.6 acid anhydrides (acid anhydride hydrolases, including helicases and GTPase), EC 3.7 carbon-carbon bonds, EC 3.8 halide bonds, EC 3.9: phosphorus-nitrogen bonds, EC 3.10: sulphur-nitrogen bonds, EC 3.11: carbon-phosphorus bonds, EC 3.12: sulfur-sulfur bonds, and EC 3.13: carbon-sulfur bonds.

In some preferred embodiments, the hydrolase is a glycoside hydrolase. These enzymes have a variety of uses including degradation of plant materials (e.g. cellulases for degrading cellulose to glucose that are used for ethanol production), food manufacturing (e.g. sugar inversion, maltodextrin production), and paper production (removing hemicelluloses from paper pulp).

In some preferred embodiments, the hydrolase is lipolase 100L (EC 3.1.1.3). It is used to synthesize pregabalin (marketed as by Pfizer as Lyrica®), an anticonvulsant drug used for neuropathic pain, anxiety disorders, and epilepsy. These conditions affect about 1% of the world's population. Lipolase 100L was found to reduce the required starting material by 39% and cut the waste per unit by 80%.

In some preferred embodiments, the hydrolase is a gamma-lactamase (e.g. EC 3.1.5.49). It is used to make Vince lactam, an intermediate for abacavir production (an antiretroviral drug for treating HIV/AIDS). It was found that changing from a stoichiometric process to a catalytic flow process reduced the number of unit operations from 17 to 12 and reduced the waste by 35%. Additionally, the use of the toxic substance cyanogen chloride is minimized.

In some preferred embodiments, the hydrolase is a Lactase (e.g. EC 3.2.1.108). These enzymes break apart lactose in milk into simple sugars to produce lactose-free milk. This important product serves approximately 15% of the world population that is lactose intolerant.

In some preferred embodiments, the hydrolase is a penicillin amidase (e.g. EC 3.5.1.11). These enzymes split penicillin into a carboxylate and 6-aminopenicillanate (6-APA). 6-APA is the core structure in natural and synthetic penicillin derivatives. These enzymes are used to produce semisynthetic penicillins tailored to fight specific infections.

In some preferred embodiments, the hydrolase is a nitralase (e.g. EC 3.5.5.1). These enzymes split nitriles into carboxyl groups. A nitralase is used to manufacture atorvastatin (marketed by Pfizer as Lipitor®). It catalyzes the reaction of meso-3-hydroxyglutaronitrile to ethyl (R)-4-cyano-3-hydroxybutyrate, the latter of which form the core of atorvastatin.

Hydrolases are discussed in the following references, incorporated herein by reference in their entirety: Anastas, P. T. Handbook of Green Chemistry. Wiley-VCH-Verlag, 2009; Dunn, Peter J., Andrew Wells, and Michael T. Williams, eds. Green chemistry in the pharmaceutical industry. John Wiley & Sons, 2010.; Martinez et al., Curr. Topics Med. Chem. 13(12):1470-90 (2010); Wells et al., Organic Process Res. Dev. 16(12):1986-1993 (2012).

In some embodiments, the invention provides hydrogen peroxide producing (HPP) enzymes. In certain embodiments, the HPP enzymes are oxidases that may be of the EX 1.1.3 subgenus. In particular embodiments, the oxidase may be EC 1.1.3.3 (malate oxidase), EC 1.1.3.4 (glucose oxidase), EC 1.1.3.5 (hexose oxidase), EC 1.1.3.6 (cholesterol oxidase), EC 1.1.3.7 (aryl-alcohol oxidase), EC 1.1.3.8 (L-gulonolactone oxidase), EC 1.1.3.9 (galactose oxidase), EC 1.1.3.10 (pyranose oxidase), EC 1.1.3.11 (L-sorbose oxidase), EC 1.1.3.12 (pyridoxine 4-oxidase), EC 1.1.3.13 (alcohol oxidase), EC 1.1.3.14 (catechol oxidase), EC 1.1.3.15 (2-hydroxy acid oxidase), EC 1.1.3.16 (ecdysone oxidase), EC 1.1.3.17 (choline oxidase), EC 1.1.3.18 (secondary-alcohol oxidase), EC 1.1.3.19 (4-hydroxymandelate oxidase), EC 1.1.3.20 (long-chain alcohol oxidase), EC 1.1.3.21 (glycerol-3-phosphate oxidase), EC 1.1.3.22, EC 1.1.3.23 (thiamine oxidase), EC 1.1.3.24 (L-galactonolactone oxidase), EC 1.1.3.25, EC 1.1.3.26, EC 1.1.3.27 (hydroxyphytanate oxidase), EC 1.1.3.28 (nucleoside oxidase), EC 1.1.3.29 (Nacylhexosamine oxidase), EC 1.1.3.30 (polyvinyl alcohol oxidase), EC 1.1.3.31, EC 1.1.3.32, EC 1.1.3.33, EC 1.1.3.34, EC 1.1.3.35, EC 1.1.3.36, EC 1.1.3.37 D-arabinono-1,4-lactone oxidase), EC 1.1.3.38 (vanillyl alcohol oxidase), EC 1.1.3.39 (nucleoside oxidase, H₂O₂ forming), EC 1.1.3.40 (D-mannitol oxidase), or EC 1.1.3.41 (xylitol oxidase).

Some embodiments of the invention may comprise hydroxylases. Hydroxylation is a chemical process that introduces a hydroxyl group (—OH) into an organic compound. Hydroxylation is the first step in the oxidative degradation of organic compounds in air. Hydroxylation plays a role in detoxification by converting lipophilic compounds into hydrophilic products that are more readily excreted. Some drugs (e.g. steroids) are activated or deactivated by hydroxylation. Hydroxylases are well-known in the art. Exemplary hydroxylases include proline hydroxylases, lysine hydroxylases, and tyrosine hydroxylases.

Some embodiments of the invention comprise Nitrilases (NIT). They are hydrolyzing enzymes (EC 3.5.5.1) that catalyze the hydrolysis of nitriles into chiral carboxylic acids with high enantiopurity and ammonia. NIT activity may be measured by monitoring the conversion of mandelonitirile into a (R)-mandelic acid. This results in a pH drop that may be monitored spectrophotometrically. Nitrilases are used to produce nicotinic acid, also known as vitamin B3 or niacin, from 3-cyanopyridine. Nicotinic acid is a nutritional supplement in foods and a pharmaceutical intermediate. Exemplary industrial uses are discussed in Gong et al., Microbial Cell Factories, 11(1), 142 (2012), incorporated herein by reference herein in its entirety.

Some embodiments of the invention comprise hydratases. They are enzymes that catalyze the addition or removal of the elements of water. Hydratases, also known as hydrolases or hydrases, may catalyze the hydration or dehydration of C—O linkages.

Some embodiments of the invention comprise oxidoreductases. These enzymes catalyze the transfer of electrons from one molecule to another. This involves the transfer of H and O atoms or electrons from one substance to another. They typically utilize NADP or NAD+ as cofactors.

In some preferred embodiments of the invention, Oxidoreductases are used for the decomposition of pollutants such as polychlorinated biphenyls and phenolic compounds, the degradation of coal, and the enhancement of the fermentation of wood hydrolysates. The invention further includes their use in biosensors and disease diagnosis.

In some preferred embodiments, the oxidoreductase is a dehydrogenase (DHO). This group of oxidoreductases oxidizes a substrate by a reduction reaction that transfers one or more hydrides (H—) to an electron acceptor, usually NAD+/NADP+ or a flavin coenzyme such as FAD or FMN. Exemplary dehydrogenases include aldehyde dehydrogenase, acetaldehyde dehydrogenase, alcohol dehydrogenase, glutamate dehydrogenase, lactate dehydrogenase, pyruvate dehydrogenase, glucose-6-phosphate dehydrogenase, glyceraldehyde-3-phosphate dehydrogenase, sorbitol dehydrogenase, isocitrate dehydrogenase, alpha-ketoglutarate dehydrogenase, succinate dehydrogenase, and malate dehydrogenase.

In some preferred embodiments, the oxidoreductase is a ketoreductase (EC 1.1.1.184), an oxidoreductase used to make atorvastatin (marketed by Pfizer as) Lipitor®. This biocatalytic process is commercially important because it substantially reduces starting materials, limits the use of organic solvents, and increases the biodegradability of the waste streams.

In some preferred embodiments, the oxidoreductase is a glucose dehydrogenase (e.g. EC 1.1.99.10). They are used by pharmaceutical companies to recycle cofactors used in drug production. They catalyze the transformation of glucose into gluconate. NADP+ is reduced to NADPH. This is used in Avastan production.

In some preferred embodiments, the oxidoreductase is P450 (EC 1.14.14.1). It is used in the pharmaceutical industry for difficult oxidations. P450 reduces the cost, inconsistency, and inefficiency associated with natural cofactors (e.g., NADPH/NADP+).

In some preferred embodiments, the oxidoreductase is a catalase such as EC 1.11.1.6. It is used in the food industry for removing hydrogen peroxide from milk prior to cheese production and for producing acidity regulators such as gluconic acid. Catalase is also used in the textile industry for removing hydrogen peroxide from fabrics.

In some preferred embodiments, the oxidoreductase is a glucose oxidase (e.g. Notatin, EC 1.1.3.4). It catalyzes the oxidation of glucose to hydrogen peroxide and D-glucono-δ-lactone. It is used, for example, to generate hydrogen peroxide as an oxidizing agent for hydrogen peroxide consuming enzymes such as peroxidase.

In some embodiments, the invention encompasses Free Radical Producing (FRP) enzymes. In some embodiments, the FRP is a peroxidase. Peroxidases are widely found in biological systems and form a subset of oxidoreductases that reduce hydrogen peroxide (H₂O₂) to water in order to oxidize a large variety of aromatic compounds ranging from phenol to aromatic amines. Peroxidases are very potent enzymes yet notoriously difficult to deploy in industrial settings due to strong inhibition in presence of excess peroxide. The invention provides increased reaction turnover and reduced inhibition. Thus, enzymes such as Horseradish Peroxidase (HRP) may be used at industrial scales.

Peroxidases belong to the sub-genus EC 1.11.1. In certain embodiments, the EC 1.11.1 enzyme is The EC 1.11.1 enzyme can be more specifically, for example, EC 1.11.1.1 (NADH peroxidase), EC 1.11.1.2 (NADPH peroxidase), EC 1.11.1.3 (fatty acid peroxidase), EC 1.11.1.4, EC 1.11.1.5 (cytochrome-c peroxidase), EC 1.11.1.6 (catalase), EC 1.11.1.7 (peroxidase), EC 1.11.1.8 (iodide peroxidase), EC 1.11.1.9 (glutathione peroxidase), EC 1.11.1.10 (chloride peroxidase), EC 1.11.1.11 (L-ascorbate peroxidase), EC 1.11.1.12 (phospholipid-hydroperoxide glutathione peroxidase), EC 1.11.1.13 (manganese peroxidase), EC 1.11.1.14 (diarylpropane peroxidase), or EC 1.11.1.15 (peroxiredoxin).

Horseradish peroxidase (EC 1.11.1.7) is a heme-containing oxidoreductase enzyme found in the roots of the horseradish plant A. rusticana. It is commonly used as a biochemical signal amplifier and tracer, as it usually acts on a chromogenic substrate together with hydrogen peroxide to produce a brightly colored product complex. It improves spectrophotometric detectability of target molecules. This characteristic of horseradish peroxidase (HRP) has been applied to permeability studies of rodent nervous system capillaries. In some embodiments of the invention, HRP is used as part of a possible remediation strategy of phenolic wastewaters due to its ability to degrade various aromatic compounds. See Duan et al., ChemPhysChem, 15(5), 974-980 (2014), incorporated by reference herein in its entirety.

In other embodiments, the peroxidase may also be further specified by function, e.g., a lignin peroxidase, manganese peroxidase, or versatile peroxidase. The peroxidase may also be specified as a fungal, microbial, animal, or plant peroxidase. The peroxidase may also be specified as a class I, class II, or class III peroxidase. The peroxidase may also be specified as a myeloperoxidase (MPO), eosinophil peroxidase (EPO), lactoperoxidase (LP), thyroid peroxidase (TPO), prostaglandin H synthase (PGHS), glutathione peroxidase, haloperoxidase, catalase, cytochrome c peroxidase, horseradish peroxidase, peanut peroxidase, soybean peroxidase, turnip peroxidase, tobacco peroxidase, tomato peroxidase, barley peroxidase, or peroxidasin. In particular embodiments, the peroxidase is horseradish peroxidase.

The lactoperoxidase/glucose oxidase (LP/GOX) antimicrobial system occurs naturally in bodily fluids such as milk, saliva, tears, and mucous (Bosch et al., J. Applied Microbiol., 89(2), 215-24 (2000)). This system utilizes thiocyanate (SCN—) and iodide (I—), two naturally occurring compounds that are harmless to mammals and higher organisms (Welk et al. Archives of Oral Biology, 2587 (2011)). LP catalyzes the oxidation of thiocyanate and iodide ions into hypothiocyanite (OSCN—) and hypoiodite (OI—), respectively, in the presence of hydrogen peroxide (H₂O₂). The H₂O₂ in this system is provided by the activity of GOX on β-D-glucose in the presence of oxygen. These free radical compounds, in turn, oxidize sulfhydryl groups in the cell membranes of microbes (Purdy, Tenovuo et al. Infection and Immunity, 39(3), 1187 (1983); Bosch et al., J. Applied Microbiol., 89(2), 215-24 (2000), leading to impairment of membrane permeability (Wan, Wang et al. Biochemistry Journal, 362, 355-362 (2001)) and ultimately microbial cell death.

Some embodiments of the invention comprise transferases. “Transferase” refers to a class of enzymes that transfer specific functional groups from one molecule to another. Examples of groups transferred include methyl groups and glycosyl groups. Transferases are used for treating substances such as chemical carcinogens and environmental pollutants. Additionally, they are used to fight or neutralize toxic chemicals and metabolites found in the human body.

In some preferred embodiments, the transferase is a transaminase. A transaminase or an aminotransferase catalyzes a reaction between an amino acid and an α-keto acid. They are important in the synthesis of amino acids. In transamination, the NH₂ group on one molecule is exchanged with the ═O from another group (e.g. a keto group) on the other molecule.

In more preferred embodiments, the transaminase is ω-transaminases (EC 2.6.1.18). It is used, among other things, to synthesize sitagliptin (marketed by Merck and Co. as Januvia®, an antidiabetic drug). Engineered ω-transaminases were found to improve biocatalytic activity by, for example, 25,000 fold, resulting in a 13% overall increase in sitagliptin yield and 19% reduction in overall process waste.

Due to their high stereoselectivity for substrates and stereospecificity for products, ω-transaminases can be utilized to make unnatural amino acids and optically pure chiral amines or keto acids (Mathew & Yun, ACS Catalysis 2(6), 993-1001 (2012)). ω-Transaminases also have applications in biocatalytic chiral resolution of active pharmaceutical intermediates, simplifying the process over conventional chemical methods. (Schatzle et al., Anal. Chem. 81(19):8244-48 (2009).) The foregoing are incorporated by reference in their entirety.

In some preferred embodiments, the transferase is a thymidylate synthetase (e.g. EC 2.1.1.45). These enzymes are used for manufacturing sugar nucleotides and oligosaccharides. They catalyze, for example, the following reaction:

5,10-methylenetetrahydrofolate+dUMP [symbol] dihydrofolate+dTMP.

In some preferred embodiments, the transferase is a glutathione S-transferase (e.g. EC 2.5.1.18). These enzymes catalyze glutathione into other tripeptides. They are used in the food industry as oxidizing agents as well as in the pharmaceutical industry to make anti-aging drugs and skin formulations.

In some preferred embodiments, the transferase is a glucokinase (e.g. EC 2.7.1.2). These enzymes facilitate the phosphorylation of glucose to glucose-6-phosphate. They are used in the food industry to reduce the glucose concentration in their production streams and as in the pharmaceutical industry to make diabetes drugs.

In some preferred embodiments, the transferase is a riboflavin kinase (e.g. EC 2.7.1.26). In a more preferred embodiment, a riboflavin kinase is used to produce flavin mononucleotide (FMN) in the food industry. FMN is an orange-red food color additive and an agent that breaks down excess riboflavin (vitamin B₂). Riboflavin kinase catalyzes, for example, the following reaction:

ATP+riboflavin [symbol] ADP+Flavin mononucleotide (FMN).

Some embodiments of the invention comprise ene reductases (EREDS). These enzymes catalyze alkene reduction in an NAD(P)H-dependent manner. Examples of ene reductases include The FMN-containing Old Yellow Enzyme (OYE) family of oxidoreductases (EC 1.6.99), clostridial enoate reductases (EnoRs, C 1.3.1.31), flavin-independent medium chain dehydrogenase/reductases (MDR; EC 1.3.1), short chain dehydrogenase/reductases (SDR; EC 1.1.1.207-8), leukotriene B4 dehydrogenase (LTD), quinone (QOR), progesterone 5b-reductase, rat pulegone reductase (PGR), tobacco double bond reductase (NtDBR), Cyanobacterial OYEs, LacER from Lactobacillus casei, Achr-OYE4 from Achromobacter sp. JA81, and Yeast OYEs.

Some embodiments of the invention comprise imine reductases (IREDS). Imine reductases (IRED) catalyze the synthesis of optically pure secondary cyclic amines. They may convert a ketone or aldehyde substrate and a primary or secondary amine substrate to form a secondary or tertiary amine product compound. Exemplary IREDs are those from Paenibacillus elgii B69, Streptomyces ipomoeae 91-03, Pseudomonas putida KT2440, and Acetobacterium woodii. IREDs are discussed in detail in Int'l Pub. No. WO2013170050, incorporated by reference herein in its entirey.

In some embodiments of the invention, the enzymes are lyases. They catalyze elimination reactions in which a group of atoms is removed from a substrate by a process other than hydrolysis or oxidation. A new double bond or ring structure often results. Seven subclasses of lyases exist. In preferred embodiments, pectin lyase is used to degrade highly esterified pectins (e.g. in fruits) into small molecules. Other preferred embodiments of the invention comprise oxynitrilases (also referred to as mandelonitrile lyase or aliphatic (R)-hydroxynitrile lyase). They cleave mandelonitrile into hydrogen cyanide+benzaldehyde.

In a preferred embodiment, the lyase is a hydroxynitrile lyase (e.g. EC 4.1.2, a mutation of a Prunus amygdalus lyase). Hydroxynitrile lyases catalyze the formation of cyanohydrins which can serve as versatile building blocks for a broad range of chemical and enzymatic reactions. They are used to improve enzyme throughput and stability at a lower pH and is used for producing clopidogrel (Plavix®). The reaction process is described in Glieder et al., Chem. Int. Ed. 42:4815 (2003), incorporated by reference herein in its entirety.

In another preferred embodiment, the lyase is 2-deoxy-D-ribose phosphate aldolase (DERA, EC 4.1.2.4). It is used for forming statin side chains, e.g. in Lipitor production.

In another preferred embodiment, the lyase is (R)-mandelonitrile lyase (HNL, EC 4.1.2.10). It is used to synthesize Threo-3-Aryl-2,3-dihydroxypropanoic acid, a precursor cyanohydrin used to produce Diltiazem. Diltiazem is a cardiac drug that treats high blood pressure and chest pain (angina). Lowering blood pressure reduces the risk of strokes and heart attacks. It is a calcium channel blocker. Ditiazem and its production are described in Dadashipour and Asano, ACS Catal. 1:1121-49 (2011) and Aehle W. 2008. Enzymes in Industry, Weiley-VCH Verlag, GmbH Weinheim, both of which are incorporated by reference in their entirety.

In another preferred embodiment, the lyase is nitrile hydratase (EC 4.2.1). It is used commercially to convert 3-cyanopyridine to nicotinamide (vitamin B3, niacinamide). It is also used in the preparation of levetiracetam, the active pharmaceutical ingredient in Keppra®.

In another preferred embodiment, the lyase is a Phenyl Phosphate Carboxylase. They are used, e.g., for phosphorylating phenol at room temperature and under sub-atmospheric CO₂ pressure. These enzymes catalyze the synthesis of 4-OH benzoic acid from phenol and CO₂ with 100% selectivity. 4-OH benzoic acid is used in the preparation of its esters. In more preferred embodiments, the enzymes are used for producing parabens that are used as preservatives in cosmetics and opthalmic solutions.

In some embodiments of the invention, the enzyme is a carbonic anhydrase (e.g. EC 4.2.1.1). Carbonic anhydrases are ubiquitous metalloenzymes present in every organism. They are among the most efficient enzymes known and serves multiple physiological roles including CO₂ exchange, pH regulation, and HCO₃⁻ secretion. Carbonic anhydrase also has potential industrial applications in CO2 sequestration and calcite production. See Lindskog & Silverman, (2000), The catalytic mechanism of mammalian carbonic anhydrases EXS 90:175-195 (W. R. Chegwidden et al. eds. 2000); In The Carbonic Anhydrases: New Horizons 7^{th} Edition pp. 175-95 (W. R. Chegwidden et al. eds. 2000); McCall et al., J. Nutrition 130:1455-1458 (2000); Boone et al., Int'l J. Chem. Engineering Volume 2013: 22-27 (2013). The foregoing are incorporated by reference in their entirety.

In some embodiments of the invention, the enzyme is an isomerase. Isomerases catalyze molecular isomerizations, i.e. reactions that convert one isomer to another. They can facilitate intramolecular rearrangements in which bonds are broken and formed or they can catalyze conformational changes. Isomerases are well known in the art.

In preferred embodiments, isomerases are used in sugar manufacturing. In more preferred embodiments, the isomerase is Glucose isomerase, EC 5.3.1.18. In other embodiments, the glucose isomerase is produced by Actinoplanes missouriensis, Bacillus coagulans or a Streptomyces species. Glucose isomerase converts D-xylose and D-glucose to D-xylulose and D-fructose, important reactions in the production of high-fructose corn syrup and in the biofuels sector.

In another preferred embodiment, the isomerase is Maleate cis-trans isomerase (EC 5.2.1.1). It catalyzes the conversion of maleic acid into fumaric acid. Fumaric acid is important for the biocatalytic production of L-aspartic acid, L-malic acid, polyester resins, food and beverage additives, and mordant for dyes.

In another preferred embodiment, the isomerase is linoleate cis-trans isomerase (EC 5.2.1.5). It catalyzes the isomerization of conjugated linoleic acid (CLA). CLA has been reported to have numerous potential health benefits for treating obesity, diabetes, cancer, inflammation, and artherogenesis. Different isomers of CLA may exert differential physiological effects. Thus, the enzyme is used to prepare single isomers.

In another preferred embodiment of the invention, the isomerase is triosephosphate isomerase (EC 5.3.1.1). It catalyzes the interconversion of D-glyceraldehyde 3-phosphate and dihydroxyacetone phosphate. In combination with transketolases or aldolases, triosephosphate isomerase is used in the stereoselective multienzyme synthesis of various sugars or sugar analogs. A preferred embodiment is the one-pot enzymatic preparation of D-xylulose 5-phosphate. This synthesis starts with the retro-aldol cleavage of fructose 1,6-biphosphate by D-fructose 1,6-biphosphate aldolase (EC 4.1.2.13). The following racemization, triosephosphate isomerase facilitates the generation of two equivalents of D-glyceraldehyde 3-phosphate that is converted into xylulose 5-phosphate by transketolase (EC 2.2.1.1)

In other embodiments of the invention, the enzyme is a Ligase. These enzymes catalyze the formation of covalent bonds joining two molecules together, coupled with the hydrolysis of a nucleoside-triphosphate. Ligases are well-known in the art and are commonly used for recombinant nucleic acid applications. In a preferred embodiment, the DNA ligase is EC 6.5.1.1.

In a preferred embodiment, the ligase is Acetyl-CoA Carboxylase (EC 6.4.1.2, ACC). ACC has a role at the junction of the lipid synthesis and oxidation pathways. It is used with the inventions disclosed herein for clinical purposes such as the production of antibiotics, diabetes therapies, obesity, and other manifestations of metabolic syndrome.

In another preferred embodiment, the ligase is Propionyl-CoA Carboxylase (PCC, EC 6.4.1.3). It catalyzes the biotin-dependent carboxylation of propionyl-CoA to produce D-methylmalonyl-CoA in the mitochondrial matrix. Methylmalyl-CoA is an important intermediate in the biosynthesis of many organic compounds as well as the process of carbon assimilation.

In some embodiments, the methods described herein use recombinant cells that express the enzymes used in the invention. Recombinant DNA technology is known in the art. In some embodiments, cells are transformed with expression vectors such as plasmids that express the enzymes. In other embodiments, the vectors have one or more genetic signals, e.g., for transcriptional initiation, transcriptional termination, translational initiation and translational termination. Here, nucleic acids encoding the enzymes may be cloned in a vector so that they are expressed when properly transformed into a suitable host organism. Suitable host cells may be derived from bacteria, fungi, plants, or animals as is well-known in the art.

Although BNCs (Level 1) provide the bulk of enzyme immobilization capability, they are sometimes too small to be easily captured by standard-strength magnets. Thus, sub-micrometric magnetic materials (Level 2) are used to provide bulk magnetization and added stability to Level 1. Commercially available free magnetite powder, with particle sizes ranging from 50-500 nm, is highly hydrophilic and tends to stick to plastic and metallic surfaces, which, over time, reduces the effective amount of enzyme in a given reactor system. In addition, powdered magnetite is extremely dense, thus driving up shipping costs. It is also rather expensive—especially at particle sizes finer than 100 nm. To overcome these limitations, low-density hybrid materials consisting of magnetite, non-water-soluble cross-linked polymers such as poly(vinylalcohol) (PVA) and carboxymethylcellulose (CMC), have been developed. These materials are formed by freeze-casting and freeze-drying water-soluble polymers followed by cross-linking. These materials have reduced adhesion to external surfaces, require less magnetite, and achieve Level 1 capture that is at least comparable to that of pure magnetite powder.

In one embodiment, the continuous macroporous scaffold has a cross-linked polymeric composition. The polymeric composition can be any of the solid organic, inorganic, or hybrid organic-inorganic polymer compositions known in the art, and may be synthetic or a biopolymer that acts as a binder. Preferably, the polymeric macroporous scaffold does not dissolve or degrade in water or other medium in which the hierarchical catalyst is intended to be used. Some examples of synthetic organic polymers include the vinyl addition polymers (e.g., polyethylene, polypropylene, polystyrene, polyacrylic acid or polyacrylate salt, polymethacrylic acid or polymethacrylate salt, poly(methylmethacrylate), polyvinyl acetate, polyvinyl alcohol, and the like), fluoropolymers (e.g., polyvinylfluoride, polyvinylidenefluoride, polytetrafluoroethylene, and the like), the epoxides (e.g., phenolic resins, resorcinol—formaldehyde resins), the polyamides, the polyurethanes, the polyesters, the polyimides, the polybenzimidazoles, and copolymers thereof. Some examples of biopolymers include the polysaccharides (e.g., cellulose, hemicellulose, xylan, chitosan, inulin, dextran, agarose, and alginic acid), polylactic acid, and polyglycolic acid. In the particular case of cellulose, the cellulose may be microbial- or algae-derived cellulose. Some examples of inorganic or hybrid organic-inorganic polymers include the polysiloxanes (e.g., as prepared by sol gel synthesis, such as polydimethylsiloxane) and polyphosphazenes. In some embodiments, any one or more classes or specific types of polymer compositions provided above are excluded as macroporous scaffolds.

### Example 1—Preparation of Macroporous Polymeric Hybrid Scaffold Powder

In order to produce the precursor solution, stock solutions of polymers were first prepared. Poly(vinylalcohol) (PVA, Sigma-Aldrich, St. Louis, Mo.), MW=89,000-98,000, 99% hydrolyzed, was dissolved to a stock concentration of 10% w/w in Milli-Q water at 70° C. HEC (Sigma-Aldrich), MW=250,000), CMC (generic low-viscosity, Sigma), and EHEC (EHM 300, Bermocoll) were each dissolved to a stock concentration of 2% w/v in Milli-Q water. Next, 1.56-3.00 g magnetite powder (Sigma-Aldrich) of two different particle size distributions (“Fine” (F), 50-100 nm and “Medium” (M), 100-500 nm) was weighed out and set aside. The amount of each reagent used was varied depending on the desired ratio of magnetite to polymer as well as the desired concentration of dry solids after freeze-drying. Excess water was added to reduce viscosity and increase the extent of ice growth and pore formation during freeze-casting.

When the solutions were ready to be freeze-cast, the magnetite was added to the polymer solutions along with solid powdered citric acid (for future PVA cross-linking step), to a final concentration of 250 mM. The mixture was immediately sonicated at 35% amplitude (⅛″ tip) for 3 min. After sonication, the solution was directly frozen in a bath of liquid nitrogen, then freeze-dried at −10° C. and 0.01 torr overnight or until dry. To initiate PVA crosslinking, the formed dry monoliths were placed in an oven at 130° C. for 60-120 minutes. Finally, the monoliths were washed with 60° C. water to remove excess crosslinker and ground in a Waring commercial blender for 30-60 seconds.

The scaffolds were cast in this example in the shape of a tubular monolith. “MO” refers to both monolithic precursor solution. The first set of numbers immediately following the MO indicate the formulation number. Thus, the above optimized monoliths are variations on the 32^{nd} monolith formulation. The second set of numbers following the hyphen indicate the dilution. Undiluted monolith (for example MO32) lacks this number, and corresponds to a total volume of 20 mL dissolving a particular fixed mass of magnetite, PVA, and CMC, as can be calculated above. MO32-30 indicates the same solid mass but dissolved in a total volume of 30 mL instead, MO32-40 indicates dilution to 40 mL, etc. The precursor solution viscosity was measured on an A&D Company Vibro Viscometer SV-10 (Toshima-ku, Tokyo, Japan) at room temperature. “Hi μ.” indicates those monoliths made with high-viscosity (˜2000-3800 cP) CMC. The lack of a label here indicates monoliths made with low-viscosity (<50 cP) CMC.

MO32 (1.875 g magnetite powder (50-100 nm), 3.125 mL 10% poly(vinyl alcohol), 3.125 mL 2% low-viscosity carboxymethylcellulose [CMC], and 13.75 mL water, crosslinked with 0.96 g citric acid)—total volume ˜20 mL. The viscosity of the precursor solution was 3.85 cP at room temperature.

MO32-30 (1.875 g magnetite powder (50-100 nm), 3.125 mL 10% poly(vinyl alcohol), 3.125 mL 2% low-viscosity carboxymethylcellulose [CMC], and 23.75 mL water, crosslinked with 0.96 g citric acid)—total volume ˜30 mL. The viscosity of the precursor solution was 2.33 cP at room temperature.

MO32-40-hi μ (1.875 g magnetite powder (50-100 nm), 3.125 mL 10% poly(vinyl alcohol), 3.125 mL 2% high-viscosity carboxymethylcellulose [CMC] (Aqualon 7H3SXFPH from Ashland), and 33.75 mL water, crosslinked with 0.96 g citric acid)—total volume ˜40 mL. The viscosity of the precursor solution was 3.65 cP at room temperature.

MO32-50-hi μ (1.875 g magnetite powder (50-100 nm), 3.125 mL 10%. poly(vinyl alcohol), 3.125 mL 2% high-viscosity carboxymethylcellulose [CMC] Aqualon 7H3SXFPH from Ashland), and 43.75 mL water, crosslinked with 0.96 g citric acid)—total volume ˜50 mL. The viscosity of the precursor solution was 3.59 cP at room temperature. The magnetite mass and fineness indicates the characteristics of Sigma-supplied magnetite used in each formulation. The mass of citric acid used to crosslink the PVA corresponds to 250 mM equivalent concentration as previously explained, and is calculated based on the mass of PVA used via the following formula (Equation 1):

m_{CA}=(m_{PVA}/0.3125)(0.02c_{CA}M_{CA}) (1)

### Where

m_{CA} is the mass of citric acid required in grams,

m_{PVA} is the total mass of PVA in solution in grams,

c_{CA} is the target citric acid concentration in mol/L (here, we used 0.25 M)

M_{CA} is the molecular mass of citric acid, 192.2 grams/mol.

The volume of magnetite and citric acid were negligible compared to the overall volume of the sample and were ignored in the calculations.

Low citric acid to polymer ratio (lower than 1:1) and duration of curing (less than 1 hour) resulted in poor crosslinking. Poorly crosslinked material are partially soluble in water and lose their pore and surface structure.

The four formulations have been scaled-up successfully to 300 mL of solution each by freezing six 50 mL tubes in parallel. Given a target total dry mass of monolith m_{T} desired, production of the solutions can be easily scaled up by applying the following formulas:

$$\begin{matrix} Magnetite {m}_{{Fe}_{3} {O}_{4}} = 5 {m}_{T} / 6 & ( 2 ) \\ PVA {m}_{PVA} = 5 {m}_{T} / 36 = {c}_{PVA , s} {V}_{PVA , s} & ( 3 ) \\ CMC {m}_{CMC} = {m}_{T} / 36 = {c}_{CMC , s} {V}_{CMC , s} & ( 4 ) \\ Water ( if dry stock polymers are used ) {V}_{W} = {fm}_{T} / 2.25 & ( 5 ) \\ Water ( if aqueous stock polymers solutions used ) {V}_{W}^{′} = \frac{{fm}_{T}}{2.25} - {V}_{PVA , s} - {V}_{CMC , s} & ( 6 )\end{matrix}$$

### Where:

TABLE 1

| | |
|---|---|
| m_{T} is the target production | VPVA, s is the volume of |
| mass in grams, | PVA stock required in mL, |
| m_{Fe}_{₃}_{O}_{₄} is the mass of mag- | VCMC, s is the volume of |
| netite required in grams, | CMC stock required in mL, |
| mPVA is the total mass of | VW is the required total volume |
| PVA required in grams, | of water in mL if dried polymer |
| mCMC is the total mass of | powders are used to prepare the |
| PVA in solution in grams, | precursor solutions, |
| cPVA, s is the stock concen- | VW‘ is the required additional |
| tration of PVA in grams/mL, | volume of water if stock solutions |
| cCMC, s is the stock concen- | at concentrations cPVA, s and |
| tration of CMC in grams/mL, | cCMC are used, and |
| | f is the dilution factor (f = 20 for |
| | undiluted, 30 for MO32-30, 40 |
| | for MO32-40, etc) |

The intact monolith were macroporous. MO32-30 had a porosity of 68.07% and MO32-50 a porosity of 67.7% with pore diameter 449 and 3.85 μm, respectively. The skeletal density was 0.86 and 0.71 g/ml, respectively, as measured by mercury porosimetry (Micromeritics, Norcross, Ga., USA).

At higher water content, more viscous polymers were used to maintain a good suspension of the particulate magnetite prior to the ice templation. The viscosity was adjusted by using water soluble polymers with lower degrees of substitution while keeping the total amount of solids constant. The solution was more viscous when the degree of substitution of the polymer was lower.

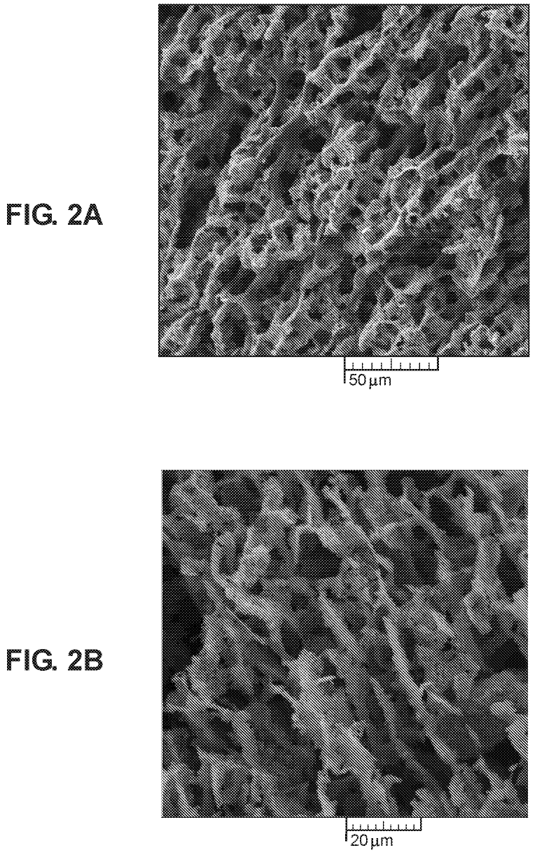

The monolith materials were mostly macroporous with submicrometric macropores (FIG. 2) but no mesopores. After grinding, the macroporosity was reduced due to the loss of macropores. The total surface area was conserved during grinding as the inner surface of the macropores became the outer surface of the particles resulting from the broken pore cells. The particle size was controlled by the intensity of the grinding and the sieving. The non-sieved powder from the monolith M32 had a measured surface area of 2.67 m²/g (Langmuir Surface Area). The non-sieved powder from the monolith M32-30 had a measured surface area of 2.8 m²/g (Langmuir Surface Area).

For a BNC loading of 50% onto MO32 powder, the calculated porosity was increased from 2.8 m²/g to 75 m²/g due the mesoporous structure of the BNCs.

The total porosity, and bulk density of the ground material, can be tuned by adjusting the quantity of water in the system, amount of cross-linkable polymers, and viscosity of the precursor solution. These parameters control the formation of the ice crystals.

To determine: the magnetic susceptibility of the materials, the magnetic moments (μ) were first measured at different magnetic field strengths (J) (i.e. a magnetic hysteresis loop experiment was performed) using a Quantum Design (San Diego, Calif., USA) Physical Property Measurement System (PPMS) unit. For comparison, magnetic behavior was also measured for pure 50-100 nm magnetite powder. These moments were then normalized for total sample mass m. It was determined that the relationship between p and II was very nearly linear (R\hat{ }2>0.985) for magnetic field strengths between −500 and 500 Oe (−39,790 A/m to 39,790 A/m). The mass magnetic susceptibility χ(m) was calculated based on the slope of the hysteresis curve in this highly linear domain, i.e. χ(m)=μ/(m*H)

The mass magnetic susceptibilities for pure 50-100 nm magnetite powder, and powdered scaffolds MO32, MO32-30, MO32-40, and MO32-50-hi μ were calculated as 9.23·10⁻⁴, 6.34·10⁻⁴, 5.63·10⁻⁴, 6.14·10⁻⁴, and 6.16·10⁻⁴ m³/kg, respectively. This is consistent with typical reported values for magnetite and other similar magnetic minerals. In addition, because the polymers have negligible magnetic response, the reported values of the hybrid material susceptibilities correspond very well with the approximate mass fraction of magnetite remaining in the scaffolds (typically ranging from 40-90 mass %).

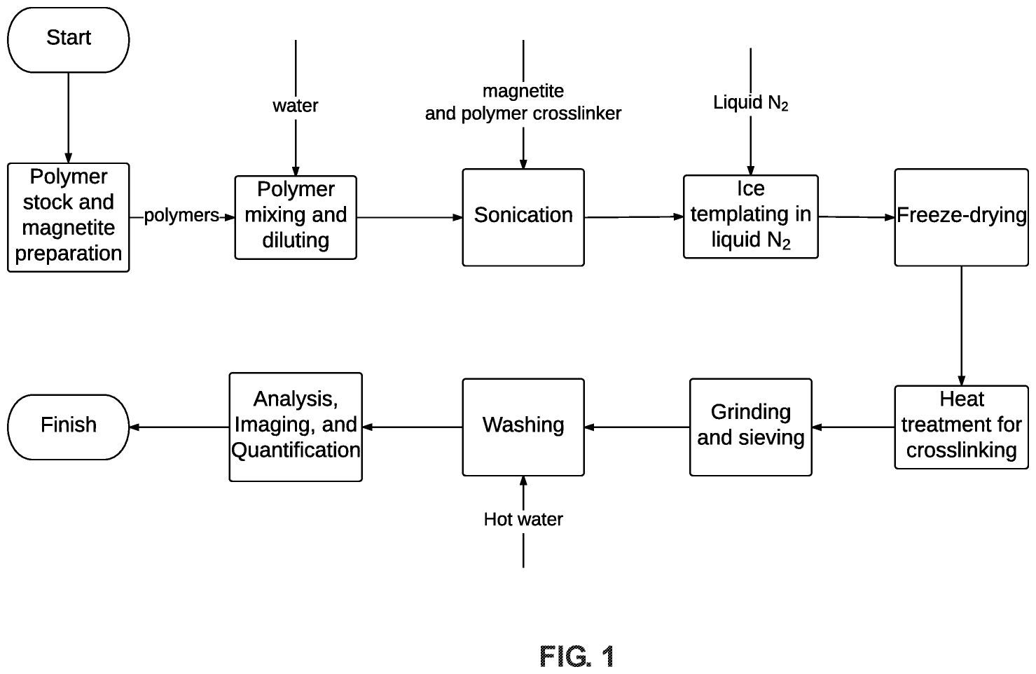

FIG. 1 shows an exemplary production process in a block diagram format for the production of the monolithic materials and ground powders. As disclosed herein, the process can encompass a greater scope of conditions and materials.

FIGS. 2-4 show scanning electron micrograph (SEM) images of monolithic materials produced under a wide variety of conditions. All monoliths depicted were freeze-cast, freeze-dried, and cross-linked at high temperature. As the ice crystals grew during freeze-casting, they produced laminar channel structures that formed thin walls of excluded materials composed of mixed polymer (smooth surfaces in the SEM images) and magnetite (small cubic crystals in the SEM images). This growth also produced macropores in the 1-50 μm range. While not wishing to be bound by theory, the higher dilution used in the precursor solution and the lower the viscosity of the precursor solution, the larger the pores will be formed.

FIG. 2A shows a scanning electron micrograph (SEM) image of magnetic scaffold MO32 (1.875 g magnetite, 3.125 mL 10% poly(vinyl alcohol), 3.125 mL 2% low-viscosity carboxymethylcellulose (CMC), and 13.75 mL excess water).

FIG. 2B shows an SEM image of magnetic scaffold MO32-50-hi μ (1.875 g magnetite, 3.125 mL 10% poly(vinyl alcohol), 3.125 mL 2% high-viscosity carboxymethylcellulose (CMC), and 43.75 mL excess water). Comparing FIGS. 2A and 2B shows an increase in apparent pore size with increasing dilution (more water) in the precursor solution.

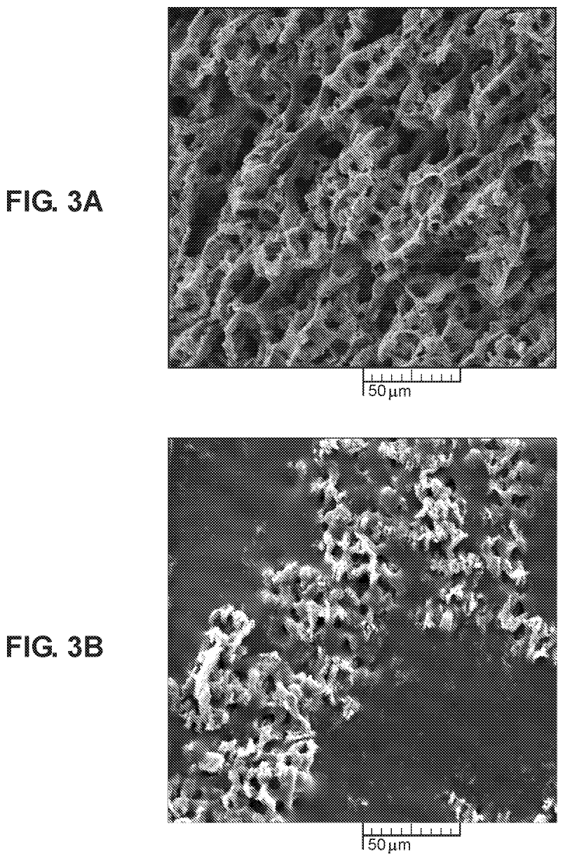

FIG. 3A shows an SEM image of magnetic scaffold MO32 (1.875 g magnetite, 3.125 mL 10% poly(vinyl alcohol), 3.125 mL 2% low-viscosity carboxymethylcellulose (CMC), and 13.75 mL excess water), containing 83% magnetite by dry solid mass.

Optimal monolith production occurred when minimal phase separation between the polymer and magnetite occurs during freeze-casting. When grinding to a powder, the porous laminar network is retained after heat treatment, grinding, and dispersing in water. FIG. 3B shows SEM image of failed magnetic scaffold MO48 (0.90 g magnetite, 11 mL 10% poly(vinyl alcohol), 3.71 mL 6% low-viscosity carboxymethylcellulose (CMC), and 23.2 mL excess water), which contained only 40% magnetite by dry solid mass. The mass ratio of poly(vinyl alcohol) to CMC was the same for both trials. Both images were taken after the scaffolds were heated to crosslink at 130° C. for one hour. Note how the monolith containing 83% magnetite (3(a)) maintained the ice-templated channel structure and pore network after being heat-treated, whereas the 40% magnetite monolith (3(b)) melted and pores fused. The reduction in magnetite content resulted in the total loss of the pore structure during the cross linking step due to phase transition and phase separation of the polymers at temperature above 100° C. The loss of porosity was also observed at a macroscopic level as the monolith shrunk significantly during the curing. In contrast, at higher concentrations of magnetite, the aligned particles acted as a scaffold on which the polymers melted as they cross-linked with the citric acid. In this condition, the macropores and overall structures of the material were preserved. This suggested that a minimum proportion of magnetite is required to act as an internal skeleton on which polymers can crosslink correctly and form macroporous, well-distributed networks.

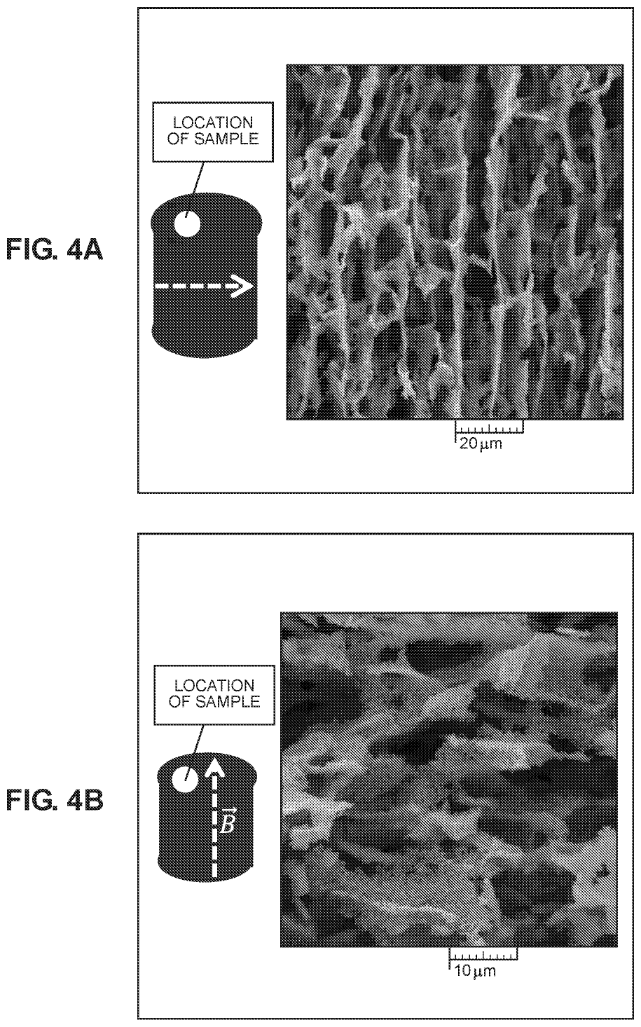

FIG. 4A shows an SEM image of magnetic scaffold MO32-40 (1.875 g magnetite, 3.125 mL 10% poly(vinyl alcohol), 3.125 mL 2% low-viscosity carboxymethylcellulose (CMC), and 33.75 mL excess water), containing 83% magnetite by dry solid mass, frozen while applying a uniform magnetic field of about 2G, perpendicular to the liquid nitrogen bath.

FIG. 4B shows an SEM image of magnetic scaffold MO32-40 (1.875 g magnetite, 3.125 mL 10% poly(vinyl alcohol), 3.125 mL 2% low-viscosity carboxymethylcellulose (CMC), and 33.75 mL excess water), containing 83% magnetite by dry solid mass, frozen while applying a uniform magnetic field of about 2G, parallel to the liquid nitrogen bath. Refer to the schematic cylinders on the left of each figure for sample locations.

The direction of channel formation and magnetite alignment can be controlled by applying an external magnetic field B (either parallel or perpendicular) to the freezing vessel. The initial orientation and alignment of the magnetite particles can constrain the ice crystal nucleation and directional growth during the freezing of the monolith. Macroscopic observations showed differences in monolith's organization of the layered materials. Parallel orientation of the external magnetic field at the time of freezing resulted in a material that was very brittle and peeling vertically. Perpendicular orientation of the external magnetic field at the time of freezing resulted in a material that was more sturdy and peeling horizontally. External magnetic fields can be used to induce preferential cleaving plans in the materials.

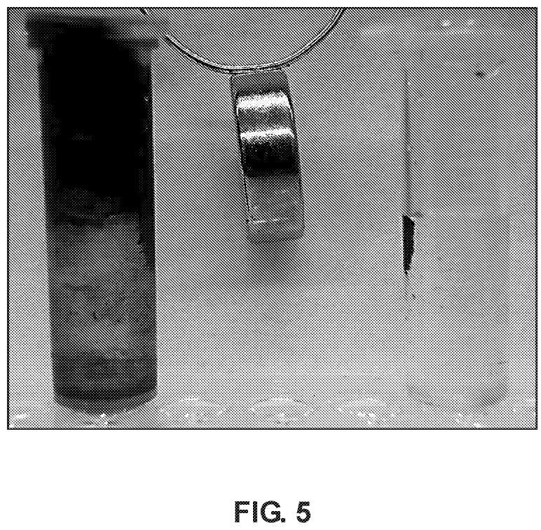

The resulting crosslinked materials were stable in solution and possessed different surface properties than the magnetite powders. FIG. 5 demonstrates the reduced surface fouling potential of the scaffolds as opposed to ordinary magnetite powder. The picture shows two tubes. The tube on the left contained 1 mL of pure magnetite powder (50-100 nm) at 2.5 mg/mL in aqueous solution. The tube on the right contained 1 mL of ground magnetic scaffold MO32, also at 2.5 mg/mL in aqueous solution. In the center was a neodymium magnet that attracted the magnetic materials in solution but not those adhering to the tube walls. Both tubes were intermittently but equally agitated over 2 months. The tube on the left showed significant fowling. The tube on the right showed virtually no fowling.

The finest monolith powders (size <100 μm) can be easily pipetted or handled by liquid transfer without loss of material or immobilized enzymes due to unspecific surface adsorption. The magnetic susceptibility of the scaffolds is dependent upon the quantity, mass, and density of the embedded magnetite.

### Example 2—Use of Magnetic Scaffolds in Biocatalysis

The powders from the ground monolithic materials were used to immobilize the BNCs and compared to regular magnetite powder for immobilized enzyme activities. Table 2 summarizes the enzymes immobilized within the BNCs, their immobilization efficiencies, and the percent effective loadings.

The total surface area of the BMC enzyme carrier (BNCs templated on powders) for a 50% loading of BNCs templated onto 50% of powder is estimated around 80 m² per gram of material where 95% of the surface is originating from the BNCs and 5% from the scaffolding material. The more BNCs are immobilized on the monolith powder, the greater the surface area and mesoporous volume.

TABLE 2

| | | Immobilization | Effective |
|---|---|---|---|
| Enzyme | BMC scaffold | efficiency | loading (%) |
| Nitrilase | Magnetite powder | 95% | 9.5 |
| | (50-100 nm) | | |
| | MO32-40 | >99% | 10 |
| ω- | Magnetite powder | 62% | 6.2 |
| Transaminase | (50-100 nm) | | |
| | MO32-40 | >99% | 10 |
| Carbonic | Magnetite powder | 95% | 9.5 |
| anhydrase | (50-100 nm) | | |
| | MO32-40 | 95% | 9.5 |
| Horseradish | Magnetite powder | >99% | 3.0 |
| peroxidase | (50-100 nm) | | |
| | MO32-40 | >99% | 3.0 |

Immobilization efficiency is defined as the ratio of mass of enzyme immobilized to the total initial mass of enzyme before immobilization. Effective loading is defined as the ratio of total initial mass of enzyme before immobilization to the total mass of magnetic scaffold used, multiplied by the immobilization efficiency. The immobilization efficiency is defined in Equation 7:

$$immobolization efficiency = {η}_{I} = \frac{{m}_{IE}}{{m}_{e}} \qquad ( 7 )$$

The effective loading is defined in Equation 8:

$$effective loading = {L}_{E} = \frac{mass enzyme actually immobilized}{total mass of magnetic supports} = \frac{{η}_{I} {m}_{E}}{{m}_{MP}} = \frac{{m}_{IE}}{{m}_{MP}} \qquad ( 8 )$$

In this text, the term loading (no qualifier) or nominal loading may also be used. These terms are distinct from the effective loading, and are defined in Equation 9:

$$( nominal ) loading = {L}_{E}^{′} = \frac{mass enzyme immobilized assuming 100 \% capture}{total mass of magnetic supports} = \frac{{m}_{E}}{{m}_{MP}} = \frac{{L}_{E}}{{η}_{I}} \qquad ( 9 )$$

where:
m_{IE} is the mass of enzyme successfully immobilized,
m_{E} is the total mass of free enzyme present initially,
m_{MP} is the total mass of all magnetic supports used—this includes the mass of the magnetite nanoparticles and that of the secondary scaffold, if applicable.
ηₗ is the immobilization efficiency, determined after protein quantification is complete,
L_{E} is the effective enzyme mass loading, and
L_{E}′ is the nominal enzyme mass loading.

### Immobilized Nitrilases

BNCs containing nitrilase (14 identical subunits each with MW=41 kDa, pI=8.1) and magnetite nanoparticles were synthesized with 20% loading (L_{E}′=0.2), then templated onto either magnetic macroporous polymeric hybrid scaffolds or pure magnetite powder, forming BMCs with 10% overall effective loading (L_{E}=0.1). The optimized immobilization condition resulted in 95% retained activity relative to the free enzyme for synthesis of nicotinic acid.

Materials and Reagents.

Recombinant nitrilase expressed in E. coli (Sigma-Aldrich catalog no. 04529, batch no. BCBL7680V), 3-cyanopyradine, o-phthaldialdehyde, 2-mercaptoethanol, BICINE-KOH, and ethanol were purchased from Sigma-Aldrich (St. Louis, Mo., USA). Hydrochloric acid, ammonium chloride, and potassium hydroxide were from Macron Fine Chemicals (Center Valley, Pa., USA) purchased at the Cornell University Chemistry Stockroom (Ithaca, N.Y., USA). Quick Start™ Bradford Protein Assay was purchased from Bio-Rad (Hercules, Calif., USA). Magnetite nanoparticles were synthesized in-house at ZYMtronix Catalytic Systems (Ithaca, N.Y., USA) as well as magnetic macroporous polymeric hybrid scaffolds, as previously described. Stock solutions were made in 18.2MΩ-cm water purified by Barnstead™ Nanopure™. Fluorescence intensity was measured in Corning Costar® 3925 black-bottom fluorescence microplates using Biotek® Synergy™ H1 plate reader operated with Gen5™ software.

Methods.

Lyophilized nitrilase was dissolved in water. O-phthaldialdehyde (OPA) stock solution (75 mM) was prepared in 100% ethanol and kept on ice or stored at 4° C. 2-mercaptoethanol (2-ME) stock solution (72 mM) was also prepared in 100% ethanol immediately prior to use. Buffered OPA/2-ME reagent was prepared by adding 450 mL of the above solutions to 9.1 mL 200 mM pH 9.0 BICINE-KOH buffer. The buffered reagent was kept on ice until just before use when it was allowed to equilibrate to room temperature (21° C.).

Nitrilase Immobilization in BNCs:

Nitrilase BNCs were synthesized with using nanoparticle suspension in water and free enzyme solution whose pHs were adjusted with 100 mM HCl and NaOH. Free nitrilase stock was diluted to 250 μg/mL and adjusted to pH 6. A 5 mL 1250 μg/mL NP suspension was sonicated using the Fisher Scientific FB-505 Sonic Dismembranator at the 40% power setting with a ¼″ probe for 1 min. The well dispersed NP suspension was adjusted to pH 3. The 20% nominal loading BNC mixture was made with equal volumes of enzyme solution and NP suspension (500 μL each), combined in a 2 mL microcentrifuge tube and mixed by inversion. The BNC mixture was gently agitated on a rotator for 10 min. Nitrilase BNC temptation on BMC scaffolds: 25 μL 50 mg/mL well-mixed BMC scaffold suspension (either magnetic macroporous polymeric hybrid or simple magnetite powder) was added to 1 mL BNC solution, then agitated gently on a rotator for 1 hour to form 10% nominal loading BMCs.

Nitrilase Reaction and Activity Determination.

Both the nitrilase (NIT) reaction and activity determination methods are based on a modification of the methods described by Banerjee, Biotechnol. Appl. Biochem. 37(3):289-293 (2003), incorporated herein by reference herein in its entirety. Briefly, nitrilase catalyzed the hydrolysis of 3-cyanopyridine to nicotinic acid by liberating ammonia. Enzyme activity was measured fluorometrically, detecting ammonia by the formation of an isoindole fluorochrome. Nitrilase reactions were run at 50° C. for 23 h in 2 mL microcentrifuge tubes using with a total reaction volume of 1 mL containing 50 mM 3-cyanopyridine, 87.5 mM BICINE-KOH, pH 9.0, and 218 nM free or immobilized nitrilase (NIT). The reaction was stopped by adding 13.35 μL 100 mM HCl to an equal volume of nitrilase reaction mix. Immobilized NIT was pelleted magnetically; its supernatant was also treated with HCl after pelleting. Activity was determined by quantification of ammonia formed in the nitrilase reaction. Buffered reagent (624 μL) was added to supernatant and was allowed to mix gently for 20 min at room temperature. After incubation, 150 μL 100 mM HCl was added to this solution to increase fluorescent signal. Fluorescence intensity was measured using 412 nm excitation, 467 nm emission with gain auto-adjusted relative to wells with highest intensity. Each fluorescence reading included an internal linear NH₄Cl standard curve (R²>0.99). A unit (U) of nitrilase activity was defined as 1 μmol NH₃ liberated per minute at 50° C. in 87.5 mM BICINE-KOH (pH 9.0).

Protein Quantification.

BMCs were pelleted magnetically and protein content in the supernatant was determined using the method in Bradford, Anal. Biochem., 72(1-2):248-254 (1976), including a linear NIT standard curve (R²>0.99). This procedure quantified the amount of unimmobilized enzyme, which allowed for determination of the immobilization efficiency and effective loading.

Results.

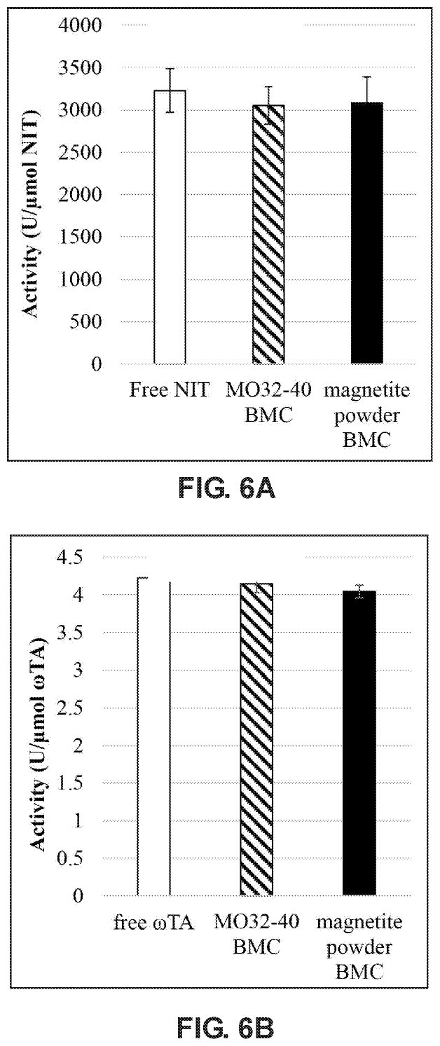

Controls showed that there was no uncatalyzed ammonia liberation. Nitrilase BNCs were templated on magnetic macroporous polymeric hybrid scaffolds with >99% immobilization efficiency for an effective loading of 10% of BMC. This was comparable to that of nitrilase BNC templated on simple magnetite powder (50-100 nm). The BMC scaffold had a 95% immobilization efficiency and a 9.5% effective loading (Table 2). The activity of the nitrilase hybrid scaffold and magnetite powder BMCs were also largely retained (>95%) relative to free nitrilase (FIG. 6A).

### Immobilized ω-Transaminase

BNCs containing ω-transaminase (MW=195 kDa) and magnetite nanoparticles were synthesized with 20% loading (L_{E}′=0.2), then templated onto either magnetic macroporous polymeric hybrid scaffolds or pure magnetite powder, forming BMCs with 10% overall effective loading (L_{E}=0.1). The optimized immobilization condition resulted in 95% retained activity relative to the free enzyme for synthesis of acetophenone from (R)-(+)-α-methylbenzylamine.

Materials and Reagents.

Recombinant ω-transaminase (ωTA) from Mycobacterium vanbaaleni expressed in E. coli, (R)-(+)-α-methylbenzylamine (MBA), sodium pyruvate, and acetophenone (AP) from Sigma (St. Louis, Mo., USA). Dimethyl sulfoxide (DMSO) was purchased from Fisher Scientific (Fair Lawn, N.J., USA). Hydrochloric acid, sodium hydroxide, and phosphate buffer salts were from Macron Fine Chemicals (Center Valley, Pa., USA). Magnetite nanoparticles as well as magnetic macroporous polymeric hybrid scaffolds were synthesized as previously described. Quick Start™ Bradford Protein Assay was purchased from Bio-Rad (Hercules, Calif., USA). Stock solutions were made with 18.2 MΩ-cm water purified by Barnstead™ Nanopure™. Absorbance was measured in triplicate in Costar™ 3635 UV-transparent microplates using Biotek Epoch™ plate reader operated with Gen5™ software.

Methods.

Lyophilized ωTA was dissolved in water. (R)-(+)-α-methylbenzylamine (MBA) stock solution was prepared by dissolving 12.78 μL MBA in 100 μL DMSO, then bringing the total volume to 10 mL with water for a final concentration of 10 mM. A 45 mM stock of sodium pyruvate was prepared by dissolving sodium pyruvate powder in water. Acetophenone stock solution was prepared by dissolving 12 μL AP in water. All stock solutions were kept on ice. Dilutions were made just before use in assays and were allowed to equilibrate to room temperature (21° C.).

ω-Transaminase Activity Assay.

ωTA activity determination methods were based on methods described by Schatzle (2009) adapted for microplates. Briefly, ωTA catalyzed the transfer of an amino-group from MBA (amine donor) to pyruvate forming AP and alanine respectively:

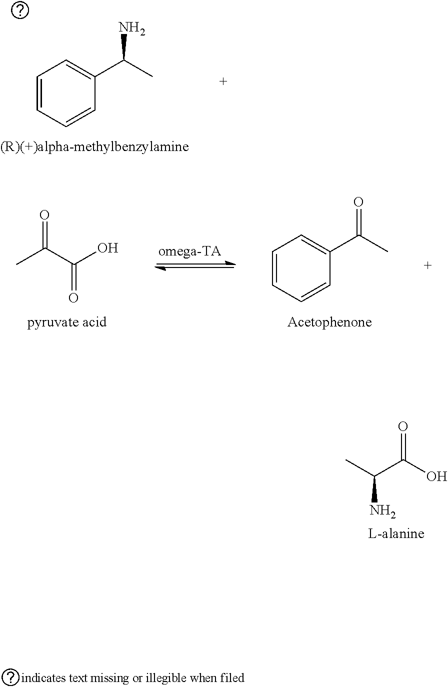

Enzyme activity was measured by the increase in absorbance at 245 nm due to the formation of AP. ωTA reactions were run at 21° C. for 1 h in 2 mL microcentrifuge tubes using with a total reaction volume of 1 mL containing 50 mM pH 8.0 phosphate buffered saline (PBS), 0.1 mM MBA, 1 mM pyruvate, and 349 nM ω-transaminase. Immobilized ωTA was pelleted magnetically and its supernatant read for absorbance. AP was quantified using a linear standard curve containing 0-0.1 mM AP and 0-0.1 mM alanine (R²>0.99). One unit (U) of ω-transaminase activity was defined as 1 μmol AP formed per minute at 21° C. in 50 mM PBS (pH 8.0).

ω-Transaminase Immobilization in BNCs:

ωTA BNCs were synthesized with using nanoparticle suspension in water and free enzyme solution whose pHs were adjusted with 100 mM HCl and NaOH. Free ωTA was diluted to 250 μg/mL and adjusted to pH 7.15. A 5 mL 1250 μg/mL NP suspension was sonicated using the Fisher Scientific FB-505 Sonic Dismembranator at the 40% power setting with a ¼″ probe for 1 min. The well dispersed NP suspension was adjusted to pH 3. The 20% nominal loading BNC mixture was made with equal volumes of enzyme solution and NP suspension (500 μL each), combined in a 2 mL microcentrifuge tube and mixed by inversion. The BNC mixture was gently agitated on a rotator for 10 min.

ω-Transaminase BNC Templation on BMC Scaffolds:

25 μL of a 50 mg/mL well-mixed BMC scaffold suspension (either magnetic macroporous polymeric hybrid or simple magnetite powder) was added to 1 mL BNC solution, then agitated gently on a rotator for 1 h to form 10% nominal loading BMCs.

Protein Quantification.

BMCs were pelleted magnetically, and protein content in the supernatant was determined using the Bradford method, including a linear ωTA standard curve (R²>0.99). This procedure quantified the amount of non-immobilized enzyme, which allowed for determination of the immobilization efficiency and effective loading.

Controls showed that there was no uncatalyzed acetophenone formation. ω-Transaminase BNCs were templated on magnetic macroporous polymeric hybrid scaffolds with >99% immobilization efficiency for an effective loading of 10% of BMC. The immobilization efficiency of the magnetic macroporous scaffold far outperformed equivalent mass of simple magnetite powder (50-100 nm) BMC scaffold (>99% vs. 62% of ωTA immobilization efficiency and 10% vs. 6.2% effective loading). See Table 2 The activity of ω-transaminase magnetic macroporous polymeric hybrid scaffold and magnetite powder BMCs were largely retained (>95%) relative to free ω-transaminase (FIG. 6B).

### Immobilized Carbonic Anhydrase

BNCs containing bovine carbonic anhydrase II (CAN) (MW=30 kDa) and magnetite nanoparticles were synthesized at 20% loading (L_{E}′=0.2), then templated onto either magnetic macroporous polymeric hybrid scaffolds or pure magnetite powder, forming BMCs with 9.5% overall effective loading (L_{E}=0.095). The optimized immobilization condition resulted in 96±9% retained activity relative to the free enzyme for dehydration of bicarbonate to carbon dioxide.

Materials and Reagents.

Carbonic anhydrase II (CA or CAN) from bovine erythrocytes, BICINE-KOH, HEPES-KOH, and 8-hydroxy-pyrene-1,3,6-trisulfonate (pyranine) were purchased from Sigma (St. Louis, Mo., USA). Hydrochloric acid, ammonium chloride, and potassium hydroxide were from Macron Fine Chemicals (Center Valley, Pa., USA) purchased at the Cornell University Chemistry Stockroom (Ithaca, N.Y., USA). Quick Start™ Bradford Protein Assay was purchased from Bio-Rad (Hercules, Calif., USA). Magnetite nanoparticles were synthesized in-house at ZYMtronix Catalytic Systems (Ithaca, N.Y., USA) as previously described as well as magnetic macroporous polymeric hybrid scaffolds, as previously described. Stock solutions were made in 18.2MΩ-cm water purified by Barnstead™ Nanopure™ Fluorescence intensity was measured in Corning Costar® 3925 black-bottom fluorescence microplates using Biotek® Synergy™ H1 plate reader, with reagent injection system, operated with Gen5™ software.

Methods.

Lyophilized CAN was dissolved in water. Reagent A contained 2 mM KHCO₃ and 0.5 mM BICINE-KOH buffer, pH 8. Reagent B contained 500 pM Carbonic Anhydrase, 100 nM pyranine, and 0.5 mM HEPES-KOH buffer, pH 6.

Carbonic Anhydrase Activity Assay.

CAN reversibly catalyzes dehydration of carbonic acid to carbon dioxide and water. The standard carbonic anhydrase activity was measured using the assay of Wilbur and Anderson (J. Biol. Chem 176:147-154 (1948)). The rate of pH decrease in a buffered CO₂-saturated solution from 8.3 to 6.3, caused by the formation of bicarbonate from carbon dioxide, is measured. An alternative fluorometric pH-based assay was used as previously described by Shingles & Moroney (Anal. Biochem. 252(1):731-737 (1997)). Briefly, pyranine is used as a fluorescent pH indicator; the increase in pH due to the dehydration of bicarbonate is reflected by an increase in fluorescence intensity. The reaction was initiated by mixing equal volumes of reagents A and B. Reagent A was added to reagent B in-microplate well with a sample injection system and fluorescence reading were begun immediately. Due to high reaction velocities, all sample reads were performed one well at a time in triplicate. Fluorescence was measured using a pH sensitive (Fₛ) and insensitive (Fᵢₛ) excitation wavelengths (466 nm and 413 nm respectively) with a 512 nm emission wavelength. Fluorescence intensity was converted to pH using a linear calibration curve of Fₛ/Fᵢₛ versus pH for buffered standards (pH 6-10) included on each plate. (Shingles & McCarty, Plant Physiol. 106(2):731-37 (1994).) One unit (U) of CAN activity was defined as the change in pH per second during the first 10 seconds of measurement under the conditions described above. The foregoing are incorporated by reference herein in its entirety

Carbonic Anhydrase Immobilization in BNCs:

CAN BNCs were formed with using nanoparticle suspension in water and free enzyme solution whose pHs were adjusted with 100 mM HCl and NaOH. Free CAN was diluted to 250 μg/mL and adjusted to pH 6. A 5 mL 1250 μg/mL NP suspension was sonicated using the Fisher Scientific FB-505 Sonic Dismembranator at the 40% power setting with a ¼″ probe for 1 min. The well dispersed NP suspension was adjusted to pH 11. The 20% nominal loading BNC mixture was made with equal volumes of enzyme solution and NP suspension (500 μL each), combined in a 2 mL microcentrifuge tube and mixed by inversion. The BNC mixture was gently agitated on a rotator for 10 min.

Carbonic Anhydrase BNC Temptation on BMC Scaffolds:

25 μL 50 mg/mL well-mixed BMC scaffold suspension (either magnetic macroporous polymeric hybrid or simple magnetite powder) was added to 1 mL BNC solution, then agitated gently on a rotator for 1 h to form 10% nominal loading BMCs.

Protein Quantification.

BMCs were pelleted magnetically, and protein content in the supernatant was determined using the Bradford method, including a linear CAN standard curve (R²>0.99), 2.5-10 μg/mL. This procedure quantified the amount of non-immobilized enzyme, which allowed for determination of the immobilization efficiency and effective loading.

Results.

The controls showed that there was no change in pH due to non-specific reactions. CAN BNCs were templated on magnetic macroporous polymeric hybrid scaffolds with 95% immobilization efficiency for an effective loading of 9.5% of BMC. This was comparable to that of CAN BNC scaffolding on simple magnetite powder (50-100 nm) BMC scaffold, which also had 95% immobilization efficiency and 9.5% effective loading (Table 2). The activity of carbonic anhydrase hybrid scaffold and magnetite powder BMCs were also mostly retained (>95%) relative to free carbonic anhydrase (FIG. 6C).

### Immobilized Horseradish Peroxidase

BNCs containing horseradish peroxidase (MW=44 kDa) and magnetite nanoparticles were synthesized with 5% nominal loading (L_{E}′=0.05) then templated onto either magnetic macroporous polymeric hybrid scaffolds or pure magnetite powder, forming BMCs with 3% overall effective loading (L_{E}=0.03). The optimized immobilization condition resulted in a four- to five-fold improvement of activity relative to the free enzyme for the complexation of phenol with 4-aminoantipyrine (4-AAP).

Materials and Reagents.

Horseradish peroxidase (HRP) from A. rusticana root, phenol, and 4-aminoantipyrine (4-AAP) were purchased from Sigma (St. Louis, Mo., USA). Hydrogen peroxide, hydrochloric acid, sodium hydroxide, and phosphate buffer salts were from Macron Fine Chemicals (Center Valley, Pa., USA) purchased at the Cornell University Chemistry Stockroom (Ithaca, N.Y., USA). Quick Start™ Bradford Protein Assay was purchased from Bio-Rad (Hercules, Calif., USA). Magnetite nanoparticles were synthesized in-house at ZYMtronix Catalytic Systems (Ithaca, N.Y., USA) as previously described, as well as magnetic macroporous polymeric hybrid scaffolds, as previously described. Stock solutions were made in 18.2MΩ-cm water purified by Barnstead™ Nanopure™. Absorbance was measured in triplicate in Costar™ 3635 UV-transparent microplates using Biotek Epoch™ plate reader operated with Gen5™ software.

Methods.

Lyophilized HRP was dissolved in water to form stock solutions. Fresh HRP reagent was prepared containing 122 mM phosphate-buffered saline (PBS) buffer, pH 7.4, 0.61 mM phenol, and 0.61 mM 4-AAP in water. This solution was stored at 4° C. and was kept in the dark until immediately before use, when it was equilibrated to reach room temperature.

Horseradish Peroxidase Immobilization in BNCs:

Horseradish peroxidase (HRP) BNCs were formed using magnetite nanoparticle (NP) suspension in water and free enzyme solution whose pH's were adjusted with 100 mM HCl and NaOH. Free HRP was diluted to 250 μg/mL and adjusted to pH 5. A 5 mL 5000 μg/mL NP suspension was sonicated using the Fisher Scientific FB-505 Sonic Dismembrator at the 40% power setting with a ¼″ probe for 1 min. The well-dispersed NP suspension was adjusted to pH 11. The 5% nominal loading BNC mixture was made with equal volumes of enzyme solution and NP suspension (525 μL each), combined in a 2 mL microcentrifuge tube and mixed by inversion. The BNC mixture was gently agitated on a rotator for 10 min.

Horseradish Peroxidase BNC Temptation on BMC Scaffolds:

250 μL of a 2.5 mg/mL well-mixed BMC scaffold suspension (either magnetic macroporous polymeric hybrid or simple magnetite powder) was added to 500 mL BNC solution, then agitated gently on a rotator for 1 h to form 3% nominal loading HRP BMCs.

Horseradish Peroxidase Activity Assay.

HRP irreversibly catalyzes the free-radical complexation of phenol and 4-AAP, using hydrogen peroxide as an initiator:

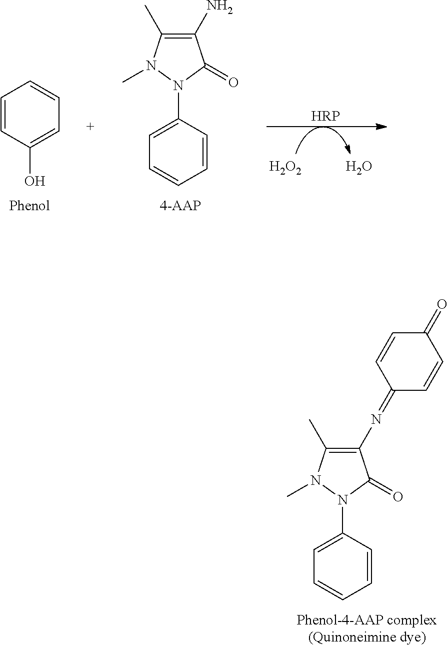 [SMILES: Oc1ccccc1 ; Cc1c(N)c(=O)n(-c2ccccc2)n1C ; "H2O2" ; O ; Cc1c(N=C2C=CC(=O)C=C2)c(=O)n(-c2ccccc2)n1C]

The resulting product is a bright pinkish-red quinoneimine dye with significant absorbance at λ=500 nm. The standard horseradish activity assay—a biocatalytic form of the Emerson-Trinder method (48^{th} Purdue University Industrial Waste Conference Proceedings. 423-430 (1993) correlates the rate of absorbance increase at λ=500 nm due to the phenolic dye product formed to the enzyme activity. HRP batch reactions for both immobilized and free HRP were run at 21° C. for 30 min in 5 mL centrifuge tubes using a total reaction volume of 3 mL containing 50 mM pH 7.4 phosphate buffered saline (PBS), 0.25 mM phenol, 0.25 mM 4-AAP, 15 nM HRP, and 0.3 mM H₂O₂ initially to begin the reaction. The batch reactions were agitated gently. At designated time points (1, 3, 30 min), triplicate absorbance readings at λ=500 nm were taken. Blanks containing the corresponding amounts of immobilized and free enzyme were also prepared to subtract the absorbance contribution of the BMCs and the background substances. Because the BMCs were very dilute in the reaction vessels, and the BMC-containing blanks had the same absorbance as free enzyme in PBS and water alone.

The product dye was quantified using extinction coefficient at 500 nm (12 mm⁻¹cm⁻¹) (Sigma Chemical Corporation and Kessey, J. (1994) Enzymatic Assay of Choline Oxidase (EC 1.1.3.17). https://www.sigmaaldrich.com/content/dam/sigma-aldrich/docs/Sigma/Enzyme_Assay/c5896enz.pdf.) One unit (U) of HRP activity was defined as 1 mmol quinoneimine dye formed per minute at 21° C. in 50 mM PBS (pH 7.4).

Protein Quantification.

BMCs were pelleted magnetically, and protein content in the supernatant was determined using the Bradford method, including a linear HRP standard curve (R²>0.99), 2.5-25 μg/mL. This procedure quantified the amount of unimmobilized enzyme, which allowed for determination of the immobilization efficiency and effective loading.

Results.

Controls showed that there was no uncatalyzed dye formation. HRP BNCs were templated on magnetic macroporous polymeric hybrid scaffolds with >99% immobilization efficiency for an effective loading of 3% of BMC. This was comparable to that of HRP BNC templated on simple magnetite powder (50-100 nm) BMC scaffold, which also had >99% immobilization efficiency and 3% effective loading (Table 2). The activities of HRP on hybrid scaffold and magnetite powder BMCs were improved four- to five-fold (400-500%) relative to free HRP (FIG. 6(d)).

### Immobilized Chloroperoxidase

BNCs containing chloroperoxidase (MW=42 kDa) and magnetite nanoparticles were synthesized with 4% nominal loading (L_{E}′=0.04) then templated onto magnetic macroporous polymeric hybrid scaffolds, forming BMCs with 0.8% overall effective loading (L_{E}=0.008). This immobilization condition resulted in a 1.6-fold improvement of enzymatic activity relative to the free enzyme for the oxidation of limonene to (1S,2S,4R)-(+)-limonene-1,2-diol, as determined by a sodium periodate-epinephrine reporter reaction.

Materials and Reagents.

Chloroperoxidase (CPO) from Caldariomyces fumago was obtained from Bio-Research Products, Inc. (North Liberty, Iowa, USA). Hydrogen peroxide, hydrochloric acid, sodium hydroxide, and phosphate buffer salts were from Macron Fine Chemicals (Center Valley, Pa., USA). (R)-limonene, glucose oxidase (GOX) from Aspergillus niger, sodium periodate (NaIO₄), catalase from bovine liver, dimethyl sulfoxide, and epinephrine were purchased from Sigma-Aldrich (St. Louis, Mo., USA). D-glucose was obtained from Alfa Aesar (Haverhill, Mass., USA). BERMOCOLL® EHM 300 substituted cellulose was obtained from AkzoNobel (Amsterdam, Netherlands). Quick Start™ Bradford Protein Assay was purchased from Bio-Rad (Hercules, Calif., USA). Magnetite nanoparticles were synthesized in-house at Zymtronix Catalytic Systems (Ithaca, N.Y., USA) as previously described, as well as magnetic macroporous polymeric hybrid scaffold MO32-40 (1.875 g of 50-100 nm magnetite in 3.125 mL of 10% poly(vinyl alcohol), 3.125 mL 2% low-viscosity carboxymethylcellulose (CMC), and 33.75 mL water, crosslinked with 250 mM citric acid). Stock solutions were made in 18.2 MΩ-cm water purified by Barnstead™ Nanopure™. Absorbance was measured in triplicate in Costar™ 3635 UV-transparent microplates using Biotek Epoch™ plate reader operated with Gen5™ software.

Methods.

Concentrated CPO solution was diluted in water to form stock solutions. Fresh primary reagent mix was prepared containing 100 mM phosphate buffer (PB) at pH 6, 100 mM glucose, 100 mM limonene emulsified with 0.016 m/v % BERMOCOLL® EHM 300, and 1 v/v % dimethyl sulfoxide (DMSO) in water. Secondary reporter mixes were prepared containing 400 μM NaIO₄ and 10 mM PB pH 6, as well as 5 mM epinephrine dissolved in HCl—the NaIO₄ and epinephrine solutions were kept separately. All reaction mixes were stored at 4° C. and kept in the dark until immediately before use, when it was equilibrated to reach room temperature.

Chloroperoxidase Immobilization in BNCs:

Chloroperoxidase (CPO) BNCs were formed using magnetite nanoparticle (NP) suspension in water and free enzyme solution. Free CPO was diluted to 100 μg/mL. A 5 mL 2500 μg/mL NP suspension was sonicated using the Fisher Scientific FB-505 Sonic Dismembrator at the 40% power setting with a ¼″ probe for 1 min. The well-dispersed NP suspension was adjusted to pH 11. The 4% nominal loading BNC mixture was made with equal volumes of enzyme solution and NP suspension (550 μL each), combined in a 2 mL microcentrifuge tube and mixed by inversion by hand for 30 s.

Chloroperoxidase BNC Temptation on BMC Scaffolds:

1 mL of BNC solution was then added to 5 mg of magnetic polymeric scaffold MO32-40, then vortexed for 1 h to form 0.8% nominal loading CPO BMCs.

Chloroperoxidase Activity Assay.

CPO catalyzes the oxidation of (R)-limonene to (1S,2S,4R)-(+)-limonene-1,2-diol, using hydrogen peroxide as initiator. To demonstrate the use of the magnetic polymeric scaffold material in a mock industrial process, relatively high (50 mM) concentration of limonene was used. To avoid excessive CPO deactivation by high peroxide concentrations, a glucose oxidase (GOX)-glucose system was implemented to produce H₂O₂ incrementally in situ. In order to quantify the diol formed, a two-step reporter reaction employing NaIO₄ and epinephrine (adrenaline) was implemented. When NaIO₄ and ephinephrine are combined alone, the resulting product is adrenochrome, a bright orange species with significant absorbance at λ=490 nm. However, if there is any diol present in the primary reaction mixture, it reduces sodium periodate to sodium iodate, lowering the amount of NaIO₄ available to the ephinephrine and thus lowering the absorbance at 490 nm. The diol in effect “competes” with epinephrine for reaction with NaIO₄. Both the primary and reporter reactions are as described in Aguila et al., Green Chemistry 10(52):647-653 (2008) and Sorouraddin et al. Biomedical Analysis 18:883-888 (1998), both of which are incorporated by reference in their entirety.

The CPO activity on limonene is directly correlated to the decrease of absorbance at λ=490 nm due to the reduction in adrenochrome formation, relative to substrate-only controls. Primary batch reactions for both immobilized and free CPO were run at 22° C. for 20 hr in 2 mL centrifuge tubes using a total reaction volume of 1 mL containing final concentrations of 50 mM pH 6 phosphate buffer, 50 mM limonene emulsified with 0.008 m/v % BERMOCOLL® EHM 300, 50 mM glucose, 50 nM CPO, 5 nM free GOX, and 0.5 v/v % DMSO. The batch reactions and appropriate controls were tumbled gently at 18 rpm in the dark. At 20 h, primary reaction mixes were diluted in preparation for the reporter step.

To quantify diol formed, 250 μL reporter reactions consisting of 400 μM NaIO₄, 10 mM, pH 6 phosphate buffer, 0.6 v/v % of the primary reaction mixture, and 100 nM catalase (to scavenge any leftover H₂O₂) were performed. This reporter-primary mixture was allowed to react for 1 minute. Then, 20 μL of 5 mM epinephrine was added per 250 μL of reporter-primary mixture. After an additional minute, absorbance was read in triplicate at a wavelength of 490 nm. Enzymatic activity was determined by the decrease of the resulting orange-colored species (adrenochrome) relative to enzyme- and substrate-free controls and an appropriate standard curve.

Protein Quantification.

BMCs were pelleted magnetically, and protein content in the supernatant was determined using the Bradford method, including a linear CPO standard curve (R²>0.99), 2.5-25 μg/mL. This procedure quantified the amount of unimmobilized enzyme, which allowed for determination of the immobilization efficiency and effective loading. In this case, a 0.8% effective loading of CPO on BMCs was determined versus a 0.8% nominal loading, indicating an enzyme capture of 100%.

Results.

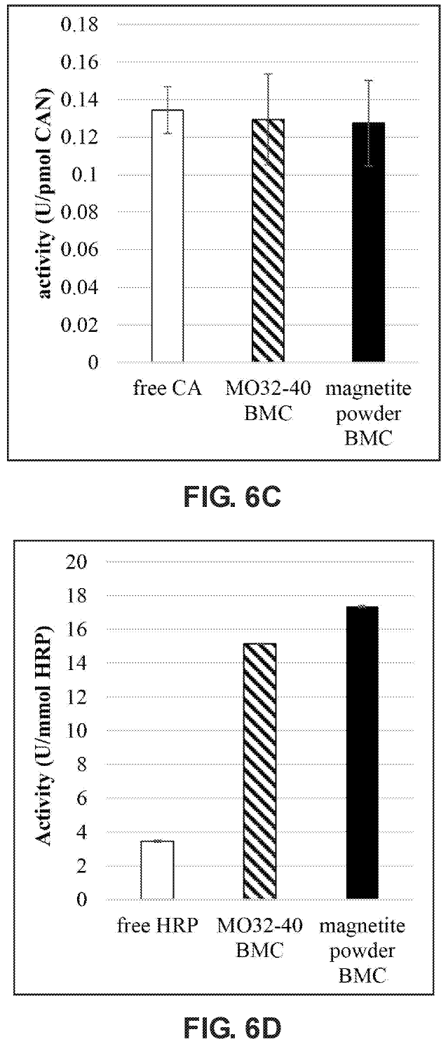

Enzyme-free controls showed that there was approximately 20% (10 mM) uncatalyzed product formation. Correcting for this baseline conversion, the conversion of limonene by CPO on hybrid scaffold BMCs was improved by 60% relative to free CPO (FIG. 7). This translates to a total (baseline+enzymatic) diol formation of about 32 mM for the immobilized CPO, versus 25 mM for free CPO. It is hypothesized that, as in FIG. 6D, peroxidase activity is enhanced on the BMCs relative to free enzyme due to higher stability and less inhibition from H₂O₂.

### Immobilized Lipase

BNCs containing lipase (MW=45 kDa) and magnetite nanoparticles were synthesized with 40% nominal loading (L_{E}′=0.40) then templated onto magnetic macroporous polymeric hybrid scaffolds, forming BMCs with 3.78% overall effective loading (L_{E}=0.038). This immobilization condition resulted in a 100% retention of activity relative to the free enzyme for the breakdown of p-nitrophenyl laurate to p-nitrophenol and laurate.

Materials and Reagents.

Lipase (LIP) from Aspergillus niger was obtained from Indo World Trading Corporation (New Delhi, India). Hydrochloric acid, sodium hydroxide, and phosphate buffer salts were from Macron Fine Chemicals (Center Valley, Pa., USA). p-nitrophenyl laurate, p-nitrophenol, bovine serum albumin (BSA), and dimethyl sulfoxide were purchased from Sigma-Aldrich (St. Louis, Mo., USA). Quick Start™ Bradford Protein Assay was purchased from Bio-Rad (Hercules, Calif., USA). Magnetite nanoparticles were synthesized as a polymeric hybrid scaffold MO32-40 (1.875 g of 50-100 nm magnetite in 3.125 mL of 10% poly(vinyl alcohol), 3.125 mL 2% low-viscosity carboxymethylcellulose (CMC), and 33.75 mL water, crosslinked with 250 mM citric acid). Stock solutions were made in 18.2MΩ-cm water purified by Barnstead™ Nanopure™. Absorbance was measured in triplicate in Costar™ 3635 UV-transparent microplates using Biotek Epoch™ plate reader operated with Gen5™ software.

Lipase Immobilization in BNCs:

Powdered lipase was dissolved in water and centrifuged. The supernatant was used to form stock solutions. Lipase (LIP) BNCs were formed using magnetite nanoparticle (NP) suspension in water and free enzyme solution. Free LIP stock was diluted to 500 μg/mL and adjusted to pH 7.4. A 5 mL 1250 μg/mL NP suspension was sonicated using the Fisher Scientific FB-505 Sonic Dismembrator at the 40% power setting with a ¼″ probe for 1 min. The well-dispersed NP suspension was adjusted to pH 3. The 40% nominal loading BNC mixture was made with equal volumes of enzyme solution and NP suspension (750 μL each), combined in a plastic deep-well microplate and mixed by vortexing for 60 s.

Lipase BNC Temptation on BMC Scaffolds:

1.5 mL of BNC solution was then added to 6.56 mg of magnetic polymeric scaffold MO32-40, then vortexed for 1 h to form 5% nominal loading LIP BMCs.

Lipase Activity Assay.

LIP catalyzes the hydrolysis of p-nitrophenyl laurate (or any analogous fatty acid derivative) to p-nitrophenol and laurate. Lipase activity was measured by the method of Gupta et al., Analytical Biochemistry 311:98-99 (2002) but modified to use p-nitrophenyl palmitate (16-carbon fatty acid), incorporated by reference herein in its entirety. In order to quantify the nitrophenol liberated, the reaction was maintained at pH 4 and absorbance readings were taken at λ=314 nm. At this pH, >99% of the nitrophenol is in the protonated form which is a light yellow species with a maximum absorbance around 314-320 nm.

The LIP activity on p-nitrophenyl laurate is directly correlated to the increase of absorbance at λ=314 nm. Batch reactions for both immobilized and free LIP were run at 45° C. for 30 min in 2 mL centrifuge tubes using a total reaction volume of 0.25 mL containing final concentrations of 100 mM pH 4 phosphate-buffered saline, 0.5 mM p-nitrophenyl laurate, 0.5 mg/mL LIP, and 2.2 v/v % DMSO. The batch reactions and appropriate controls were vortexed gently. At 30 min, triplicate absorbance readings at λ=314 nm were taken. Enzymatic activity was compared to the enzyme- and substrate-free controls and an appropriate nitrophenol standard curve at pH 4.

Protein Quantification.

BMCs were pelleted magnetically, and protein content in the supernatant was determined using the Bradford method, including a linear BSA standard curve (R²>0.99), 2.5-10 μg/mL. This procedure quantified the amount of unimmobilized enzyme, which allowed for determination of the immobilization efficiency and effective loading. In this case, a 3.78% effective loading of LIP on BMCs was determined versus a 5% nominal loading, indicating an enzyme capture of 75.6%.

Results.

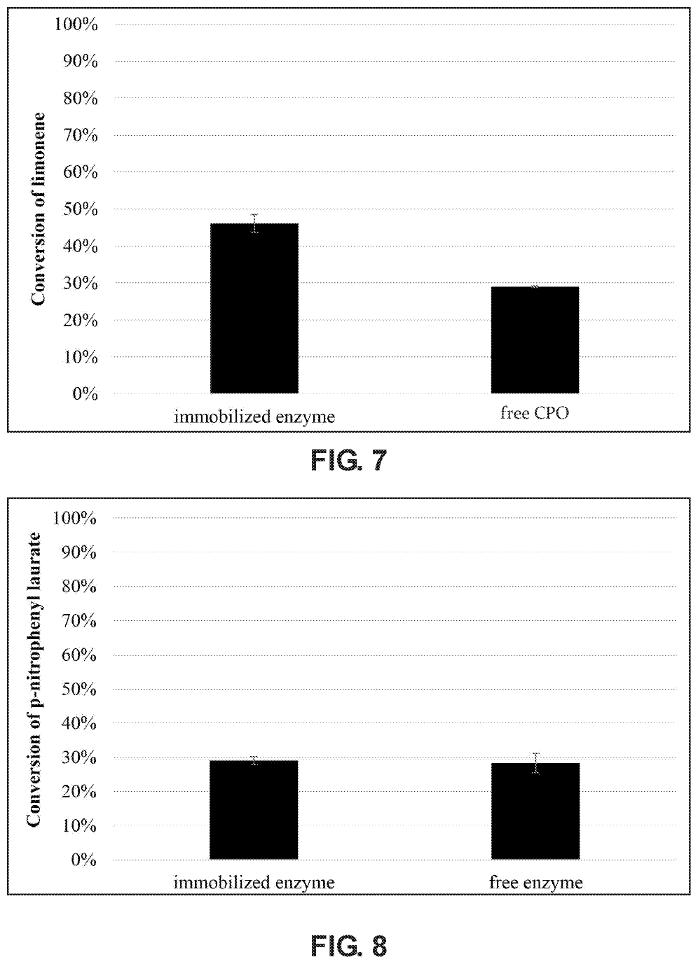

Enzyme-free controls indicated that there was approximately 4.2% (21 μM) uncatalyzed product formation. Correcting for this baseline conversion, the conversion of p-nitrophenyl laurate by LIP on hybrid scaffold BMCs was retained relative to free LIP (FIG. 8). This translates to a total (baseline+enzymatic) nitrophenol formation of about 170 μM for both immobilized and free CPO, demonstrating that the immobilization method and material described here does not appear to adversely affect the activity of the lipase used.

All publications and patent documents disclosed or referred to herein are incorporated by reference in their entirety. The foregoing description has been presented only for purposes of illustration and description. This description is not intended to limit the invention to the precise form disclosed. It is intended that the scope of the invention be defined by the claims appended hereto.

1. A magnetic macroporous polymeric hybrid scaffold, comprising a cross-linked water-insoluble polymer and an approximately uniform distribution of embedded magnetic microparticles (MMP); wherein said polymer comprises polyvinyl alcohol (PVA); wherein said MMPs are about 50-500 nm in size; wherein said scaffold comprises pores of about 1 to about 50 μm in size; wherein said scaffold comprises about 20% to 95% w/w MMP; wherein said scaffold comprises an effective surface area for incorporating bionanocatalysts (BNC) that is about total 1-15 m²/g; wherein the total effective surface area for incorporating the enzymes is about 50 to 200 m²/g; wherein said scaffold has a bulk density of between about 0.01 and about 10 g/ml; and wherein said scaffold has a mass magnetic susceptibility of about 1.0×10⁻³ to about 1×10⁻⁴ m³ kg⁻¹.

2. The magnetic macroporous polymeric hybrid scaffold of claim 1 comprising a contact angle for said scaffold with water that is about 0-90 degrees.

3. The magnetic macroporous polymeric hybrid scaffold of claim 1, further comprising a polymer selected from the group consisting of polyethylene, polypropylene, poly-styrene, polyacrylic acid, polyacrylate salt, polymethacrylic acid, polymethacrylate salt, polymethyl methacrylate, polyvinyl acetate, polyvinylfluoride, polyvinylidenefluoride, polytetrafluoroethylene, a phenolic resin, a resorcinol formaldehyde resin, a polyamide, a polyurethane, a polyester, a polyimide, a polybenzimidazole, cellulose, hemicellulose, carboxymethyl cellulose (CMC), 2-hydroxyethylcellulose (HEC), ethylhydroxyethyl cellulose (EHEC), xylan, chitosan, inulin, dextran, agarose, alginic acid, sodium alginate, polylactic acid, polyglycolic acid, a polysiloxane, a polydimethylsiloxane, and a polyphosphazene.

4. The magnetic macroporous polymeric hybrid scaffold of claim 3, wherein said scaffold comprises PVA and CMC.

5. The magnetic macroporous polymeric hybrid scaffold of claim 3, wherein said scaffold comprises PVA and alginate.

6. The magnetic macroporous polymeric hybrid scaffold of claim 3, wherein said scaffold comprises PVA and HEC.

7. The magnetic macroporous polymeric hybrid scaffold of claim 3, wherein said scaffold comprises PVA and EHEC.

8. The magnetic macroporous polymeric hybrid scaffold of claim 1, wherein said scaffold is formed in the shape of a monolith.

9. The magnetic macroporous polymeric hybrid scaffold of claim 1, wherein said scaffold is formed in a shape suited for a particular biocatalytic process.

10. The magnetic macroporous polymeric hybrid scaffold of claim 1, wherein said scaffold is in the form of a powder, wherein said powder comprises particles of about 150 to about 1000 μm in size.

11. The magnetic macroporous polymeric hybrid scaffold of claim 1, further comprising a bionanocatalyst (BNC).

12. The magnetic macroporous polymeric hybrid scaffold of claim 11, wherein said BNC comprises a magnetic nanoparticle (MNP) and an enzyme selected from the group consisting of hydrolases, hydroxylases, hydrogen peroxide producing enzymes (HPP), nitralases, hydratases, dehydrogenases, transaminases, ene reductases (EREDS), imine reductases (IREDS), oxidases, oxidoreductases, peroxidases, oxynitrilases, isomerases, and lipases.

13. A method of preparing a water-insoluble macroporous polymeric hybrid scaffold, comprising
a. mixing a water-soluble polymer with water and magnetic microparticles (MMP) to form a suspension of about 3 to 50 cP;
b. adding a cross-linking reagent to said mixture;
c. ultra-sonicating said mixture;
d. freezing said mixture at a temperature of about −200 to 0 degrees Celsius;
e. freeze drying said mixture; and
f. cross-linking said water-soluble polymer;
wherein said cross-linking step results in water-insoluble polymers.

14. The method of claim 13, wherein said cross-linking step is accomplished by exposure to ultraviolet light, heating said mixture at a temperature of about 60 to 500 degrees Celsius, or a combination thereof.

15. The method of claim 13, further comprising the step of applying a magnetic field after said ultra-sonication step to in order to organize said MMPs by alignment of the magnetic moments of said MMPs.

16. The method of claim 13 wherein said water-soluble polymer is polyvinyl alcohol (PVA).

17. The method of claim 13, further comprising a polymer selected from the group consisting of polyethylene, polypropylene, poly-styrene, polyacrylic acid, polyacrylate salt, polymethacrylic acid, polymethacrylate salt, polymethyl methacrylate, polyvinyl acetate, polyvinylfluoride, polyvinylidenefluoride, polytetrafluoroethylene, a phenolic resin, a resorcinol formaldehyde resin, a polyamide, a polyurethane, a polyester, a polyimide, a polybenzimidazole, cellulose, hemicellulose, carboxymethyl cellulose (CMC), 2-hydroxyethylcellulose, ethylhydroxyethyl cellulose, xylan, chitosan, inulin, dextran, agarose, alginic acid, sodium alginate, polylactic acid, polyglycolic acid, a polysiloxane, a polydimethylsiloxane, and a polyphosphazene.

18. The method of claim 17, wherein said polymers comprise PVA and CMC.

19. The method of claim 17, wherein said polymers comprise PVA and alginate.

20. The method of claim 17, wherein said polymers comprise PVA and HEC.

21. The method of claim 17, wherein said polymers comprise PVA and EHEC.

22. The method of claim 13, wherein said cross-linking reagent is selected from the group consisting of citric acid, all calcium salts, 1,2,3,4-butanetetracarboxylic acid (BTCA), glutaraldehyde, and poly(ethylene glycol).

23. The method of claim 22, wherein said cross-linking reagent is citric acid.

24. The method of claim 13, wherein said freezing step results in a water-soluble macroporous polymeric hybrid scaffold that is in the shape of a monolith.

25. The method of claim 13, wherein said freezing step results in a water-soluble macroporous polymeric hybrid scaffold that is in a shape suited for a particular biocatalytic process.

26. The method of claim 13, further comprising grinding said water-insoluble macroporous polymeric hybrid scaffold into a powder of about 10 to about 1000 μm in size.

27. The method of claim 13 any one of claims 13 to 23, wherein said water-insoluble macroporous polymeric hybrid scaffold is shaped into beads of about 500 to about 5000 μm in size.

28. A method of catalyzing a reaction between a plurality of substrates, comprising exposing said substrates to the magnetic macroporous polymeric hybrid scaffold of claim 11 under conditions in which said BNC catalyzes said reaction between said substrates.

29. The method of claim 28, wherein said reaction is used in the manufacture of a pharmaceutical product.

30. The method of claim 28, wherein said reaction is used in the manufacture of a medicament.

31. The method of claim 28, wherein said reaction is used in the manufacture of a food product.

32. The method of claim 28, wherein said reaction is used in the manufacture of a garment.

33. The method of claim 28, wherein said reaction is used in the manufacture of a detergent.

34. The method of claim 28, wherein said reaction is used in the manufacture of a fuel product.

35. The method of claim 28, wherein said reaction is used in the manufacture of a biochemical product.

36. The method of claim 28, wherein said reaction is used in the manufacture of a paper product.

37. The method of claim 28, wherein said reaction is used in the manufacture of a plastic product.

38. The method of claim 28, wherein said reaction is used in a process for removing a contaminant from a solution.

39. The method of claim 38, wherein said solution is an aqueous solution.

40. A magnetic macroporous polymeric hybrid scaffold, comprising a cross-linked water-insoluble polymer and an approximately uniform distribution of embedded magnetic microparticles (MMP); wherein said MMPs are about 50-500 nm in size; wherein said scaffold comprises pores of about 1 to about 50 μm in size; wherein said scaffold comprises about 20% to 95% w/w MMP; wherein said scaffold comprises an effective surface area for incorporating bionanocatalysts (BNC) that is about total 1-15 m²/g; wherein the total effective surface area for incorporating the enzymes is about 50 to 200 m²/g; wherein said scaffold has a bulk density of between about 0.01 and about 10 g/ml; and wherein said scaffold has a mass magnetic susceptibility of about 1.0×10⁻³ to about 1×10⁻⁴ m³ kg⁻¹.

41. The magnetic macroporous polymeric hybrid scaffold of claim 20, wherein said scaffold comprises a polymer selected from the group consisting of polyethylene, polypropylene, poly-styrene, polyacrylic acid, polyacrylate salt, polymethacrylic acid, polymethacrylate salt, polymethyl methacrylate, polyvinyl acetate, polyvinylfluoride, polyvinylidenefluoride, polytetrafluoroethylene, a phenolic resin, a resorcinol formaldehyde resin, a polyamide, a polyurethane, a polyester, a polyimide, a polybenzimidazole, cellulose, hemicellulose, carboxymethyl cellulose (CMC), 2-hydroxyethylcellulose (HEC), ethylhydroxyethyl cellulose (EHEC), xylan, chitosan, inulin, dextran, agarose, alginic acid, sodium alginate, polylactic acid, polyglycolic acid, a polysiloxane, a polydimethylsiloxane, and a polyphosphazene.

42. The magnetic macroporous polymeric hybrid scaffold of claim 40, further comprising a bionanocatalyst (BNC).

43. The magnetic macroporous polymeric hybrid scaffold of claim 42, wherein said BNC comprises a magnetic nanoparticle (MNP) and an enzyme selected from the group consisting of hydrolases, hydroxylases, hydrogen peroxide producing enzymes (HPP), nitralases, hydratases, dehydrogenases, transaminases, ene reductases (EREDS), imine reductases (IREDS), oxidases, oxidoreductases, peroxidases, oxynitrilases, isomerases, and lipases.
